(19) United States
(12) Patent Application Publication
(10) Pub. No.: US 2014/0330004 A1
(43) Pub. Date: Nov. 6, 2014

(54) OLIGONUCLEOTIDE

(71) Applicant: Kyowa Hakko Kirin Co., Ltd., Tokyo (JP)

(72) Inventors: Fumikazu SHINOHARA, Tokyo (JP); Asana MAKINO, Tokyo (JP); Junichiro YAMAMOTO, Tokyo (JP); Taiji OASHI, Tokyo (JP); Michihiko SUZUKI, Tokyo (JP); Jun-ichi SAITO, Tokyo (JP)

(73) Assignee: Kyowa Hakko Kirin Co., Ltd., Tokyo (JP)

(21) Appl. No.: 13/972,128

(22) Filed: Dec. 6, 2013

Related U.S. Application Data
(60) Provisional application No. 61/695,566, filed on Aug. 31, 2012.

(51) Int. Cl. C12N 15/113

(52) U.S. Cl. CPC C12N 15/113
USPC 536/24.5

(57) ABSTRACT

An oligonucleotide, which has a nucleotide residue or a nucleoside residue represented by formula (I) at the 5′ end thereof, wherein the nucleotide residue or the nucleoside residue binds to an adjacent nucleotide residue via the oxygen atom at position 3, or the like;

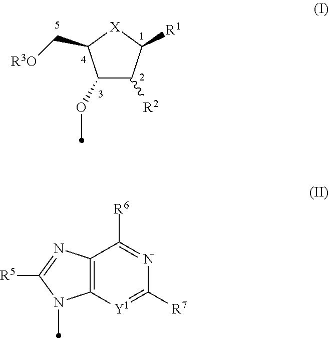

wherein X represents an oxygen atom or the like, R¹ represents formula (II) wherein Y¹ represents a nitrogen atom or the like, R⁵ represents halogen or the like, R⁶ and R⁷ may be the same or different, and each represents a hydrogen atom or the like, and R³ represents a hydrogen atom or the like.

### FIELD OF INVENTION

The present invention relates to an oligonucleotide having an unnatural nucleotide residue or an unnatural nucleoside residue introduced at the 5′ end, and the like.

### BACKGROUND ART

A small interfering RNA (hereinafter referred to as siRNA) is involved in the RNA interference (hereinafter referred to as RNAi) and is an RNA having a function as a guide for interfering with the expression of a target gene (Nature, vol. 411, No. 6836, pp. 494-498). An siRNA can selectively interfere with the expression of a protein, the expression of which is regulated by a messenger RNA (mRNA), through cleavage of the mRNA, and therefore, the application thereof to pharmaceuticals has been expected, and at present, there is also known an siRNA under clinical trials (Nature Reviews Cancer, vol. 11, pp. 59-67, 2011).

An siRNA is generally incorporated into a complex called an RNA induced silencing complex (RISC), and then exhibits its function. A main constituent component of the RISC is a protein called Argonaute 2 (AGO2), and AGO2 is known to bind to an siRNA in the RNAi pathway and cleave an mRNA (Trends in Biochemical Sciences, vol. 35, No. 7, pp. 368-376, 2010). The siRNA incorporated into the RISC is cleaved at the sense strand side and processed into a single strand of only the antisense strand, and thereafter, binds to a target mRNA which is complementary to the antisense strand. The target mRNA is cleaved by an RNAse domain in the AGO2, and resulting in interfering with the expression of the protein (Silence, vol. 1, page 3, 2010).

Further, in recent years, a conformational analysis of hAGO2 MID/AMP complex and hAGO2 MID/UMP complex (Nature, vol. 465, pp. 818-822, 2010) and a conformational analysis for a complex of hAGO2 and an RNA oligonucleotide (Science, vol. 336, page 25, 2012) have also been reported.

On the other hand, in recent years, a structural analysis of proteins particularly using an X-ray has been actively carried out, and there have been many reports of attempts to elucidate a mode of binding between a protein and a compound targeting the protein at the atomic level on the basis of the obtained structural information and to design a compound which fits the structure (Journal of Postgraduate Medicine, vol. 55, pp. 301-304, 2009).

However, although a possibility of avoiding an off-target effect using an oligonucleotide containing an unnatural nucleotide (Patent Document 1) and a possibility of enhancing the activity of an siRNA by improving the affinity for AGO2 (Non-Patent Document 1) have been suggested, a specific method for enhancing the knockdown activity by improving the affinity for AGO2 has not been disclosed yet.

### PRIOR ART DOCUMENTS

### Patent Document

[Patent Document 1] WO2011/119674

### Non-Patent Document

[Non-Patent Document 11] Molecular Therapy—Nucleic Acids vol. 1, page e5, 2012

### SUMMARY OF THE INVENTION

### Problems to be Solved by the Invention

An object of the present invention is to provide an oligonucleotide having an unnatural nucleotide residue or an unnatural nucleoside residue introduced at the 5′ end for improving the affinity for AGO2, and the like.

### Means for Solving the Problems

The present invention relates to the following (1) to (48).

(1) An oligonucleotide, which has a nucleotide residue or a nucleoside residue represented by formula (I) at the 5′ end thereof, wherein the nucleotide residue or the nucleoside residue binds to an adjacent nucleotide residue via the oxygen atom at position 3:

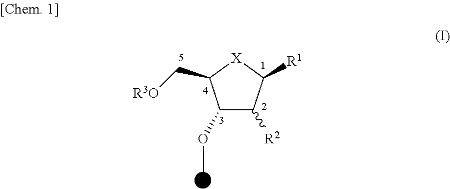

{wherein X represents an oxygen atom, a sulfur atom, a selenium atom, or NR⁴ (wherein R⁴ represents a hydrogen atom, optionally substituted lower alkyl, optionally substituted lower alkenyl, optionally substituted lower alkynyl, optionally substituted lower alkanoyl, optionally substituted lower alkylsulfonyl, optionally substituted aralkyl, optionally substituted aryl, optionally substituted aroyl, or optionally substituted aromatic heterocyclic carbonyl),

R¹ represents formula (II):

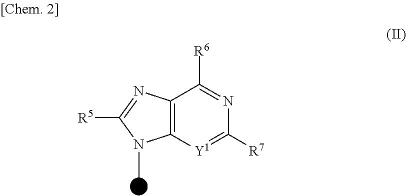

{wherein Y¹ represents a nitrogen atom or CR⁸ [wherein R⁸ represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkanoyl, optionally substituted lower alkylthio, optionally substituted aryl, optionally substituted aralkyl, an optionally substituted aromatic heterocyclic group, optionally substituted aromatic heterocyclic alkyl, —NR^{9a}R^{9b} (wherein R^{9a} and R^{9b} may be the same or different, and each represents a hydrogen atom or optionally substituted lower alkyl), or —CONR^{9c}R^{9d} (wherein R^{9c} and R^{9d} may be the same or different, and each represents a hydrogen atom or optionally substituted lower alkyl)],

R⁵ represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkylthio, optionally substituted lower alkanoyl, optionally substituted aryl, an optionally substituted aromatic heterocyclic group, —NR^{10a}R^{10b} (wherein R^{10a} and R^{10b} have the same definitions as R^{9a} and R^{9b} described above, respectively), or —CONR^{10c}R^{10d} (wherein R^{10c} and R^{10d} have the same definitions as R^{9c} and R^{9d} described above, respectively),

R⁶ and R⁷ may be the same or different, and each represents a hydrogen atom, halogen, cyano, carboxy, optionally substituted lower alkyl, optionally substituted lower alkenyl, optionally substituted lower alkynyl, optionally substituted lower alkoxy, optionally substituted lower alkylthio, optionally substituted lower alkanoyl, optionally substituted lower alkoxycarbonyl, optionally substituted aryl, optionally substituted aralkyl, optionally substituted aryloxy, optionally substituted arylthio, optionally substituted aroyl, an optionally substituted aromatic heterocyclic group, optionally substituted aromatic heterocyclic alkyl, optionally substituted aromatic heterocyclicoxy, optionally substituted aromatic heterocyclicthio, optionally substituted aromatic heterocyclic carbonyl, —NR^{11a}R^{11b} (wherein R^{11a} and R^{11b} may be the same or different, and each represents a hydrogen atom, optionally substituted lower alkyl, optionally substituted lower alkenyl, optionally substituted lower alkynyl, optionally substituted aryl, optionally substituted aralkyl, optionally substituted aromatic heterocyclic alkyl, optionally substituted lower alkanoyl, optionally substituted lower alkylsulfonyl, optionally substituted aroyl, optionally substituted lower arylsulfonyl, an optionally substituted aromatic heterocyclic group, optionally substituted aromatic heterocyclic carbonyl, or optionally substituted aromatic heterocyclic sulfonyl), —CONR^{11c}R^{11d} (wherein R^{11c} and R^{11d} may be the same or different, and each represents a hydrogen atom, optionally substituted lower alkyl, optionally substituted aryl, or an optionally substituted aromatic heterocyclic group), —NHCONR^{11e}R^{11f} (wherein R^{11e} and R^{11f} may be the same or different, and each represents a hydrogen atom, optionally substituted lower alkyl, optionally substituted aralkyl, optionally substituted aromatic heterocyclic alkyl, optionally substituted aryl, or an optionally substituted aromatic heterocyclic group), or —NHCO₂R^{11g} (wherein R^{11g} represents optionally substituted lower alkyl, optionally substituted aralkyl, optionally substituted aromatic heterocyclic alkyl, optionally substituted aryl, or an optionally substituted aromatic heterocyclic group), provided that when Y¹ is a nitrogen atom, R⁵ is a hydrogen atom, R⁶ is —NR^{11a}R^{11b}, and R⁷ is a hydrogen atom, R^{11a} and R^{11b} are not a hydrogen atom at the same time), formula (III):

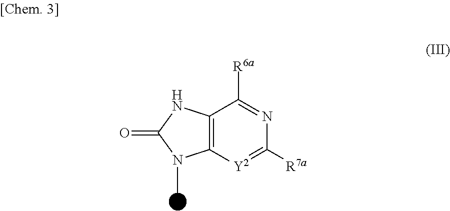

(wherein Y², R^{6a}, and R^{7a} have the same definitions as Y¹, R⁶, and R⁷ described above, respectively), or formula (IV):

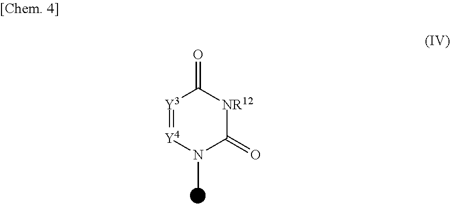

[wherein R¹² represents a hydrogen atom, optionally substituted lower alkyl, optionally substituted lower alkenyl, optionally substituted lower alkynyl, optionally substituted aralkyl, optionally substituted aromatic heterocyclic alkyl, optionally substituted lower alkanoyl, or optionally substituted lower alkylsulfonyl,

--- represents a single bond or a double bond,

provided that when --- represents a single bond,

Y³ represents NR^{13a} (wherein R^{13a} represents a hydrogen atom, optionally substituted lower alkyl, optionally substituted lower alkenyl, optionally substituted lower alkynyl, optionally substituted aralkyl, optionally substituted aromatic heterocyclic alkyl, optionally substituted lower alkanoyl, or optionally substituted lower alkylsulfonyl), or CR^{14a}R^{14b} (wherein R^{14a} and R^{14b} may be the same or different, and each represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkanoyl, optionally substituted lower alkyl thio, optionally substituted lower alkylamino, optionally substituted di-lower alkylamino, optionally substituted lower alkylcarbamoyl, optionally substituted di-lower alkylcarbamoyl, optionally substituted aryl, optionally substituted aralkyl, an optionally substituted aromatic heterocyclic group, or optionally substituted aromatic heterocyclic alkyl), and

Y⁴ represents NR^{13b} (wherein R^{13b} has the same definition as R^{13a} described above) or CR^{14c}R^{14d} (wherein R^{14c} and R^{14d} have the same definitions as R^{14a} and R^{14b} described above, respectively),

provided that when --- represents a double bond,

Y³ represents a nitrogen atom or CR^{14e} (wherein R^{14e} has the same definition as R^{14a} described above) and

Y⁴ represents a nitrogen atom or CR^{14f} (wherein R^{14f} has the same definition as R^{14a} described above), but R¹² is a hydrogen atom, and when Y³ is CR^{14e} and Y⁴ is CR^{14f}, R^{14e} and R^{14f} are not a hydrogen atom at the same time],

R² represents a hydrogen atom, hydroxy, halogen, or optionally substituted lower alkoxy, and

R³ represents a hydrogen atom or

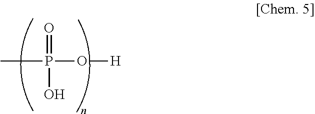

(wherein n represents 1, 2, or 3)}.

(2) The oligonucleotide according to the above (1), wherein X is an oxygen atom.

(3) The oligonucleotide according to the above (1) or (2), wherein R¹ is formula (II).

(4) The oligonucleotide according to the above (3), wherein Y¹ is a nitrogen atom.

(5) The oligonucleotide according to the above (3) or (4), wherein R⁵ is halogen, optionally substituted lower alkyl, optionally substituted lower alkenyl, optionally substituted lower alkynyl, optionally substituted lower alkoxy, optionally substituted lower alkanoyl, optionally substituted aryl, an optionally substituted aromatic heterocyclic group, —NR^{10a}R^{10b} (wherein R^{10a} and R^{10b} have the same definitions as described above, respectively), or —CONR^{10c}R^{10d} (wherein R^{10c} and R^{10d} have the same definitions as described above, respectively).

(6) The oligonucleotide according to the above (3) or (4), wherein R⁵ is halogen.

(7) The oligonucleotide according to any one of the above (3) to (6), wherein R⁶ is optionally substituted lower alkyl, optionally substituted lower alkenyl, optionally substituted lower alkynyl, optionally substituted aryl, an optionally substituted aromatic heterocyclic group, or —NR^{11a}R^{11b} (wherein R^{11a} and R^{11b} have the same definitions as described above, respectively).

(8) The oligonucleotide according to any one of the above (3) to (6), wherein R⁶ is —NR^{11a}R^{11b} (wherein R^{11a} and R^{11b} have the same definitions as described above, respectively).

(9) The oligonucleotide according to any one of the above (3) to (6), wherein R⁶ is amino.

(10) The oligonucleotide according to any one of the above (3) to (9), wherein R⁷ is a hydrogen atom.

(11) The oligonucleotide according to the above (1) or (2), wherein R¹ is formula (III).

(12) The oligonucleotide according to the above (1) or (2), wherein R¹ is formula (IV).

(13) The oligonucleotide according to the above (12), wherein R¹² is a hydrogen atom.

(14) The oligonucleotide according to the above (13), wherein --- is a double bond, Y³ is CR^{14e} (wherein R^{14e} has the same definition as described above), and Y⁴ is CR^{14f} (wherein R^{14f} has the same definition as described above).

(15) The oligonucleotide according to the above (14), wherein either one of R^{14e} and R^{14f} is a hydrogen atom.

(16) The oligonucleotide according to any one of the above (1) to (15), wherein R³ is a hydrogen atom.

(17) The oligonucleotide according to any one of the above (1) to (15), wherein R³ is

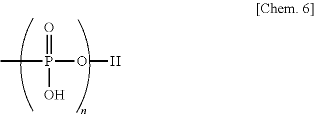

(wherein n has the same definition as described above).

(18) The oligonucleotide according to the above (17), wherein n is 1.

(19) The oligonucleotide according to the above (17), wherein n is 2.

(20) The oligonucleotide according to the above (17), wherein n is 3.

(21) An oligonucleotide, which has a nucleotide residue or a nucleoside residue represented by formula (IA) at the 5′ end thereof, wherein the nucleotide residue or the nucleoside residue binds to an adjacent nucleotide residue via the oxygen atom at position 3:

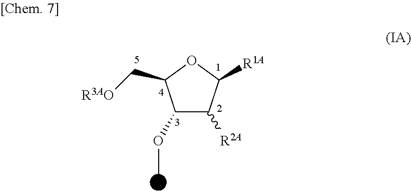

[wherein R^{1A} represents formula (IIA):

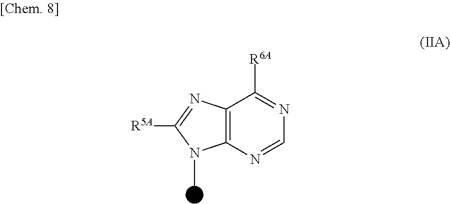

(wherein R^{5A} and R^{6A} have the same definitions as R⁵ and R⁶ described above, respectively), formula (IIIA):

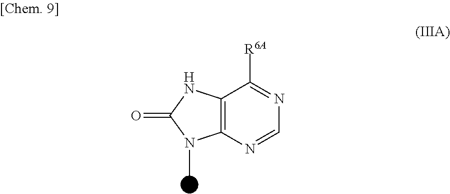

(wherein R^{6A1} has the same definition as R⁶ described above), or formula (IVA):

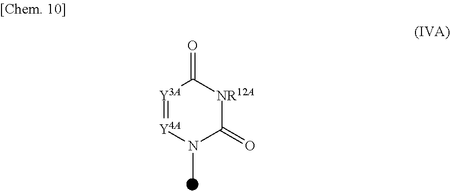

(wherein --- has the same definition as described above, R^{12A}, Y^{3A}, and Y^{4A} have the same definitions as R¹², Y³, and Y⁴ described above, respectively),

R^{2A} represents a hydrogen atom, hydroxy, halogen, or optionally substituted lower alkoxy, and

R^{3A} represents a hydrogen atom or

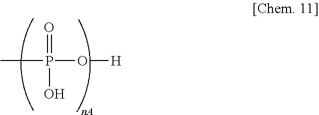

(wherein nA has the same definition as n described above)].

(22) The oligonucleotide according to the above (21), wherein R^{1A} is formula (IIA).

(23) The oligonucleotide according to the above (22), wherein R^{5A} is halogen, carbamoyl, optionally substituted lower alkyl, optionally substituted lower alkenyl, optionally substituted lower alkynyl, optionally substituted lower alkoxy, optionally substituted lower alkanoyl, optionally substituted lower alkylamino, optionally substituted di-lower alkylamino, optionally substituted lower alkylcarbamoyl, optionally substituted di-lower alkylcarbamoyl, optionally substituted aryl, or an optionally substituted aromatic heterocyclic group.

(24) The oligonucleotide according to the above (22), wherein R^{5A} is halogen.

(25) The oligonucleotide according to any one of the above (22) to (24), wherein R^{6A} is optionally substituted lower alkyl, optionally substituted lower alkenyl, optionally substituted lower alkynyl, optionally substituted aryl, an optionally substituted aromatic heterocyclic group, or NR^{11a}R^{11b} (wherein R^{11a} and R^{11b} have the same definitions as described above, respectively).

(26) The oligonucleotide according to any one of the above (22) to (24), wherein R^{6a} is NR^{11a}R^{11b} (wherein R^{11a} and R^{11b} have the same definitions as described above, respectively).

(27) The oligonucleotide according to any one of the above (22) to (24), wherein R^{6A} is amino.

(28) The oligonucleotide according to the above (21), wherein R^{1A} is formula (IVA).

(29) The oligonucleotide according to the above (28), wherein --- is a double bond, Y^{3A} is CR^{14e} (wherein R^{14e} has the same definition as described above), and Y^{4A} is CR^{14f} (wherein R^{14f} has the same definition as described above).

(30) The oligonucleotide according to the above (29), wherein R^{14e} and R^{14f} are each a hydrogen atom.

(31) The oligonucleotide according to any one of the above (21) to (30), wherein R^{3A} is a hydrogen atom.

(32) The oligonucleotide according to any one of the above (21) to (30), wherein R^{3A} is

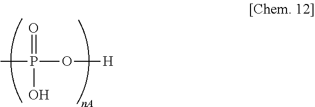

(wherein nA has the same definition as described above).

(33) The oligonucleotide according to the above (32), wherein nA is 1.

(34) The oligonucleotide according to the above (32), wherein nA is 2.

(35) The oligonucleotide according to the above (32), wherein nA is 3.

(36) The oligonucleotide according to any one of the above (1) to (35), which has a length of 10 to 80 bases.

(37) The oligonucleotide according to any one of the above (1) to (35), which has a length of 20 to 50 bases.

(38) The oligonucleotide according to any one of the above (1) to (35), which has a length of 20 to 30 bases.

(39) The oligonucleotide according to any one of the above (1) to (35), which has a length of 21 to 25 bases.

(40) The oligonucleotide according to any one of the above (1) to (39), which comprises two oligonucleotide strands.

(41) The oligonucleotide according to any one of the above (1) to (39), which comprises one oligonucleotide strand.

(42) The oligonucleotide according to any one of the above (1) to (41), wherein the oligonucleotide is an siRNA.

(43) A method for improving the knockdown activity against a target mRNA of an oligonucleotide for treating a disease, comprising:

(i) a step of selecting an oligonucleotide having a knockdown activity against a messenger RNA (mRNA) encoding a protein involved in the disease for treating the disease; and

(ii) a step of introducing a nucleotide residue or a nucleoside residue represented by formula (I) as a nucleotide residue or a nucleoside residue at the 5′ end of the oligonucleotide selected in the above (i):

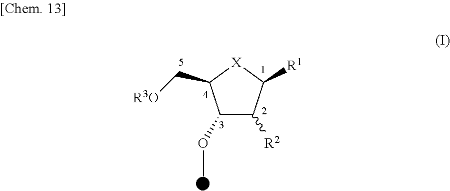

(wherein X, R¹, R², and R³ have the same definitions as described above, respectively).

(44) The method for improving the knockdown activity against a target messenger RNA (mRNA) of an oligonucleotide according to the above (43), wherein the oligonucleotide is a small interfering RNA (siRNA).

(45) A nucleotide or a salt thereof or a nucleoside or a salt thereof, which is for use in improving the knockdown activity against a target mRNA of an oligonucleotide by being introduced at the 5′ end of the oligonucleotide, and is represented by formula (Ia):

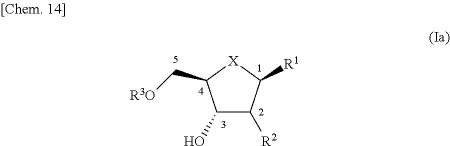

(wherein X, R¹, R², and R³ have the same definitions as described above, respectively).

(46) The nucleotide or a salt thereof or a nucleoside or a salt thereof according to the above (45), wherein the oligonucleotide is a small interfering RNA (siRNA).

(47) Use of an oligonucleotide for the manufacture of a target protein expression inhibitor, which has a nucleotide residue or a nucleoside residue represented by formula (I) at the 5′ end:

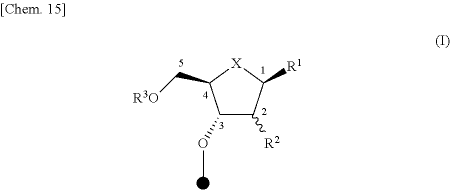

(wherein X, R¹, R², and R³ have the same definitions as described above, respectively).

(48) The use according to the above (47), wherein the oligonucleotide is a small interfering RNA (siRNA).

### Advantageous Effects of the Invention

According to the present invention, an oligonucleotide having an unnatural nucleotide residue or an unnatural nucleoside residue introduced at the 5′ end for improving the affinity for AGO2, and the like are provided.

### BRIEF DESCRIPTION OF DRAWINGS

FIG. 1 is a view for comparing the levels of luciferase luminescence between an siRNA having 8-bromo-2′-deoxyadenosine monophosphate at the 5′ end of the antisense strand and an siRNA having adenosine monophosphate with respect to luciferase-targeting siRNAs (concentration: 100 pmol/L). The vertical axis represents the ratio of luminescence in the case of using each siRNA when the amount of luminescence in a negative control group is taken as 1.

FIG. 2 is a view for comparing the levels of luciferase luminescence between an siRNA having 8-bromo-2′-deoxyadenosine monophosphate at the 5′ end of the antisense strand (874-BrdA) and an siRNA having adenosine (874-A), guanosine (874-G), cytidine (874-C), or uridine (874-U) with respect to luciferase-targeting siRNAs. The vertical axis represents the ratio of luminescence in the case of using each siRNA when the amount of luminescence in a negative control group is taken as 1. In the abscissa axis, A, G, C, U, and BrdA denote 874-A, 874-G, 874-C, 874-U, and 874-BrdA, respectively. The results of the test performed by setting the concentration of each test compound to 3.2 pmol/L, 16 pmol/L, 80 pmol/L, and 400 pmol/L are shown.

FIG. 3 is a view for comparing the expression levels of GAPDH mRNA between an siRNA having 8-bromo-2′-deoxyadenosine monophosphate at the 5′ end of the antisense strand and an siRNA having adenosine monophosphate with respect to GAPDH-targeting siRNAs. The vertical axis represents the relative expression level of GAPDH mRNA when the GAPDH mRNA level in a negative control group is taken as 1. The abscissa axis represents respective siRNAs, more specifically, it represents siRNAs having adenosine monophosphate at the 5′ end of the antisense strand (217-A, 278-A, 516-A, 624-A, 715-A, 816-A, 936-A, 1096-A, and 1134-A) on the left side, and siRNAs having 8-bromo-2′-deoxyadenosine monophosphate at the 5′ end of the antisense strand (217-BrdA, 278-BrdA, 516-BrdA, 624-BrdA, 715-BrdA, 816-BrdA, 936-BrdA, 1096-BrdA, and 1134-BrdA) on the right side.

### MODE FOR CARRYING OUT THE INVENTION

Hereinafter, a compound represented by formula (Ia) refers to Compound (Ia).

In the definitions of the respective groups in formulae (I), (II), (III), (IV), (TA), (IIA), (IIIA), (IVA), and (la),

(i) the halogen means each atom of fluorine, chlorine, bromine, and iodine.

(ii) Examples of the lower alkyl and the lower alkyl moieties of the lower alkoxy, the lower alkoxycarbonyl, the lower alkylamino, the di-lower alkylamino, the lower alkylcarbamoyl, the di-lower alkylcarbamoyl, the lower alkylthio, and the lower alkylsulfonyl include linear or branched alkyl each having 1 to 10 carbon atoms. Specific examples thereof include methyl, ethyl, propyl, isopropyl, butyl, isobutyl, sec-butyl, tert-butyl, pentyl, isopentyl, neopentyl, hexyl, heptyl, octyl, nonyl, decyl, and the like. The two lower alkyl moieties of the di-lower alkylamino and the di-lower alkylcarbamoyl may be the same or different.

(iii) The alkylene moieties of the aralkyl and the aromatic heterocyclic alkyl have the same definitions as groups in which one hydrogen atom is removed from the lower alkyl described in the above (ii).

(iv) Examples of the lower alkenyl include linear or branched alkenyl each having 2 to 10 carbon atoms. Specific examples thereof include vinyl, allyl, 1-propenyl, isopropenyl, methacryl, butenyl, crotyl, pentenyl, hexenyl, heptenyl, octenyl, nonenyl, decenyl, and the like.

(v) Examples of the lower alkynyl include linear or branched alkynyl each having 2 to 10 carbon atoms. Specific examples thereof include ethynyl, propargyl, butynyl, pentynyl, hexynyl, heptynyl, octynyl, nonynyl, decynyl, and the like.

(vi) Examples of the lower alkanoyl include linear or branched lower alkanoyl each having 1 to 8 carbon atoms. Specific examples thereof include formyl, acetyl, propionyl, butyryl, isobutyryl, valeryl, isovaleryl, pivaloyl, hexanoyl, heptanoyl, octanoyl, and the like.

(vii) Examples of the aryl and the aryl moieties of the aralkyl, the aroyl, the aryloxy, the arylthio, and the arylsulfonyl include aryl each having 6 to 14 carbon atoms. Specific examples thereof include phenyl, naphthyl, indenyl, anthryl, and the like.

(viii) Examples of the aromatic heterocyclic group and the aromatic heterocyclic group moieties of the aromatic heterocyclic alkyl, the aromatic heterocyclic carbonyl, the aromatic heterocyclicoxy, the aromatic heterocyclicthio, and the aromatic heterocyclic sulfonyl include a 5- or 6-membered monocyclic aromatic heterocyclic group having at least one atom selected from a nitrogen atom, an oxygen atom, and a sulfur atom, a bicyclic or tricyclic fused-ring aromatic heterocyclic group in which 3- to 8-membered rings are fused and at least one atom selected from a nitrogen atom, an oxygen atom, and a sulfur atom is contained, and the like. Specific examples thereof include pyridyl, pyrazinyl, pyrimidinyl, pyridazinyl, oxopyridazinyl, quinolyl, isoquinolyl, phthalazinyl, quinazolinyl, quinoxalinyl, naphthyridinyl, cinnolinyl, pyrrolyl, pyrazolyl, imidazolyl, triazinyl, triazolyl, tetrazolyl, thienyl, furyl, thiazolyl, isothiazolyl, oxazolyl, isoxazolyl, oxadiazolyl, thiadiazolyl, indolyl, isoindolyl, indazolyl, benzofuryl, isobenzofuryl, benzothienyl, benzoimidazolyl, benzotriazolyl, benzothiazolyl, benzoxazolyl, pyrazolopyridyl, pyrazolopyrimidinyl, purinyl, dibenzofuranyl, dibenzoazepinyl, and the like.

(ix) Examples of the substituents of the optionally substituted lower alkyl, the optionally substituted lower alkenyl, the optionally substituted lower alkynyl, the optionally substituted lower alkoxy, the optionally substituted lower alkoxycarbonyl, the optionally substituted lower alkanoyl, the optionally substituted lower alkylamino, the optionally substituted di-lower alkylamino, the optionally substituted lower alkylcarbamoyl, the optionally substituted di-lower alkylcarbamoyl, the optionally substituted lower alkylthio, and the optionally substituted lower alkylsulfonyl, which may be the same or different and in number of, for example, 1 to 3, include substituents selected from the group consisting of halogen, hydroxy, sulfanyl, nitro, cyano, carboxy, carbamoyl, C₃₋₈ cycloalkyl, an aliphatic heterocyclic group, C₁₋₁₀ alkoxy, C₃₋₈ cycloalkoxy, C₆₋₁₄ aryloxy, C₇₋₁₆ aralkyloxy, C₁₋₈ alkanoyloxy, C₇₋₁₅ aroyloxy, C₁₋₁₀ alkylsulfanyl, —NR^{X}R^{Y} (wherein R^{X} and R^{Y} may be the same or different, and each represents a hydrogen atom, C₁₋₁₀ alkyl, C₃₋₈ cycloalkyl, C₆₋₁₄ aryl, an aromatic heterocyclic group, C₇₋₁₆ aralkyl, C₁₋₈ alkanoyl, C₇₋₁₅ aroyl, C₁₋₁₀ alkoxycarbonyl, or C₁₋₁₀ aralkyloxycarbonyl), C₁₋₈ alkanoyl, C₇₋₁₅ aroyl, C₁₋₁₀ alkoxycarbonyl, C₆₋₁₄ aryloxycarbonyl, C₁₋₁₀ alkylcarbamoyl, and di-C₁₋₁₀ alkylcarbamoyl, and the like. The substituents of the optionally substituted lower alkenyl, the optionally substituted lower alkynyl, the optionally substituted lower alkoxy, the optionally substituted lower alkoxycarbonyl, the optionally substituted lower alkanoyl, the optionally substituted lower alkylamino, the optionally substituted di-lower alkylamino, the optionally substituted lower alkylcarbamoyl, the optionally substituted di-lower alkylcarbamoyl, the optionally substituted lower alkylthio, and the optionally substituted lower alkylsulfonyl may be substituents selected from the group consisting of C₆₋₁₄ aryl and an aromatic heterocyclic group in addition to the above-described substituents.

(x) Examples of the substituents of the optionally substituted aryl, the optionally substituted aralkyl, the optionally substituted aryloxy, the optionally substituted arylthio, the optionally substituted arylsulfonyl, the optionally substituted aroyl, the optionally substituted aromatic heterocyclic group, the optionally substituted aromatic heterocyclic alkyl, the optionally substituted aromatic heterocyclic carbonyl, the optionally substituted aromatic heterocyclicoxy, the optionally substituted aromatic heterocyclicthio, and the optionally substituted aromatic heterocyclic sulfonyl, which may be the same or different and in number of, for example, 1 to 3, include substituents selected from the group consisting of halogen, hydroxy, sulfanyl, nitro, cyano, carboxy, carbamoyl, C₁₋₁₀ alkyl, trifluoromethyl, C₃₋₈ cycloalkyl, C₆₋₁₄ aryl, an aliphatic heterocyclic group, an aromatic heterocyclic group, C₁₋₁₀ alkoxy, C₃₋₆ cycloalkoxy, C₆₋₁₄ aryloxy, C₇₋₁₆ aralkyloxy, C₁₋₈ alkanoyloxy, C₇₋₁₅ aroyloxy, C₁₋₁₀ alkylsulfanyl, —NR^{Xa}R^{Ya} (wherein R^{Xa} and R^{Ya} may be the same or different, and each represents a hydrogen atom, C₁₋₁₀ alkyl, C₃₋₈ cycloalkyl, C₆₋₁₄ aryl, an aromatic heterocyclic group, C₇₋₁₆ aralkyl, C₁₋₈ alkanoyl, C₇₋₁₅ aroyl, C₁₋₁₀ alkoxycarbonyl, or C₇₋₁₆ aralkyloxycarbonyl), C₁₋₈ alkanoyl, C₇₋₁₅ aroyl, C₁₋₁₀ alkoxycarbonyl, C₆₋₁₄ aryloxycarbonyl, C₁₋₁₀ alkylcarbamoyl, and di-C₁₋₁₀ alkylcarbamoyl, and the like.

Examples of the C₁₋₁₀ alkyl and the C₁₋₁₈ alkyl moieties of the C₁₋₁₀ alkoxy, the C₁₋₁₀ alkylsulfanyl, the C₁₋₁₀ alkoxycarbonyl, the C₁₋₁₀ alkylcarbamoyl, and the di-C₁₋₁₀ alkylcarbamoyl include the groups exemplified as the lower alkyl described above. The two C₁₋₁₀ alkyl moieties of the di-C₁₋₁₀ alkylcarbamoyl may be the same or different.

Examples of the C₁₋₈ alkanoyl and the C₁₋₈ alkanoyl moiety of the C₁₋₈ alkanoyloxy include the groups exemplified as the lower alkanoyl described above.

Examples of the C₃₋₈ cycloalkyl and the C₃₋₈ cycloalkyl moiety of the C₃₋₈ cycloalkoxy include cyclopropyl, cyclobutyl, cyclopentyl, cyclohexyl, cycloheptyl, cyclooctyl, and the like.

Examples of the C₆₋₁₄ aryl and the aryl moieties of the C₆₋₁₄ aryloxy, the C₇₋₁₅ aroyl, the C₇₋₁₅ aroyloxy, and the C₆₋₁₄ aryloxycarbonyl include the groups exemplified as the aryl described above.

Examples of the aryl moieties of the C₇₋₁₆ (aralkyloxy, the C₇₋₁₆ aralkyl, and the C₇₋₁₆ aralkyloxycarbonyl include the groups exemplified as the aryl described above. Examples of the alkylene moiety include C₁₋₁₀ alkylene, and more specifically include groups in which one hydrogen atom is removed from the groups exemplified as the lower alkyl described above.

Examples of the aliphatic heterocyclic group include a 5- or 6-membered monocyclic aliphatic heterocyclic group having at least one atom selected from a nitrogen atom, an oxygen atom, and a sulfur atom, a bicyclic or tricyclic fused-ring aliphatic heterocyclic group in which 3- to 8-membered rings are fused and at least one atom selected from a nitrogen atom, an oxygen atom, and a sulfur atom is contained, and the like. Specific examples thereof include aziridinyl, azetidinyl, pyrrolidinyl, piperidino, piperidinyl, azepanyl, 1,2,5,6-tetrahydropyridyl, imidazolidinyl, pyrazolidinyl, piperazinyl, homopiperazinyl, pyrazolinyl, oxiranyl, tetrahydrofuranyl, tetrahydro-2H-pyranyl, 5,6-dihydro-2H-pyranyl, oxazolidinyl, morpholino, morpholinyl, thioxazolidinyl, thiomorpholinyl, 2H-oxazolyl, 2H-thioxazolyl, dihydroindolyl, dihydroisoindolyl, dihydrobenzofuranyl, benzimidazolidinyl, dihydrobenzoxazolyl, dihydrobenzothioxazolyl, benzodioxolinyl, tetrahydroquinolyl, tetrahydroisoquinolyl, dihydro-2H-chromanyl, dihydro-1H-chromanyl, dihydro-2H-thiochromanyl, dihydro-1H-thiochromanyl, tetrahydroquinoxalinyl, tetrahydroquinazolinyl, dihydrobenzodioxanyl, and the like.

The halogen and the aromatic heterocyclic group have the same definitions as described above, respectively.

The oligonucleotide of the present invention means a polymer or an oligomer consisting of a nucleotide residue or a nucleoside residue.

The oligonucleotide of the present invention includes both of a single-stranded oligonucleotide and a double-stranded oligonucleotide. In the case of a double-stranded oligonucleotide, the base lengths of the respective oligonucleotide strands may be different. Further, the double-stranded oligonucleotide may contain one or more mismatched base pairs. In addition, a complex formed of three or more oligonucleotide strands is also included in the oligonucleotide of the present invention.

Examples of the double-stranded oligonucleotide include double-stranded DNAs such as structural genes, double-stranded RNAs such as small, molecule RNAs including siRNAs and miRNAs, and the like, however, the present invention is not limited thereto.

Examples of the single-stranded oligonucleotide include antisense oligonucleotides, microRNAs, aptamers, antagomirs, single-stranded RNAi agents (such as siRNAs), and the like, however, the present invention is not limited thereto.

The length of the oligonucleotide of the present invention is preferably 10 to 100 bases, more preferably 10 to 80 bases, further more preferably 10 to 50 bases, particularly preferably 20 to 50 bases, and the most preferably 20 to 30 bases.

In the oligonucleotide of the present invention, in addition to the nucleotide residue or the nucleoside residue at the 5′ end, further one or more nucleotide residues may be modified. Such modification may be contained in any site of a base, a sugar, and a phosphate.

A base-modified nucleotide may be any as long as it is a nucleotide in which a part or the whole of the chemical structure of a base of the nucleotide is modified with an arbitrary substituent or is substituted with an arbitrary atom, and examples thereof include a nucleotide in which an oxygen atom in a base is substituted with a sulfur atom, a nucleotide in which a hydrogen atom in a base is substituted with alkyl having 1 to 10 carbon atoms, a nucleotide in which methyl in a base is substituted with hydrogen or alkyl having 2 to 10 carbon atoms, and a nucleotide in which amino is protected by a protective group such as an alkyl group having 1 to 10 carbon atoms, alkanoyl having 1 to 8 carbon atoms, or the like.

A sugar moiety-modified nucleotide may be any as long as it is a nucleotide in which a part or the whole of the chemical structure of a sugar of the nucleotide is modified with an arbitrary substituent or is substituted with an arbitrary atom, however, a 2′-modified nucleotide is preferably used. Examples of the 2′-modified nucleotide include a 2′-modified nucleotide in which the 2′-OH of a ribose is substituted with a substituent selected from the group consisting of a hydrogen atom, —OR, —R, —R′, —SH, —SR, amino, —NHR, —NR₂, N₃, cyano, and halogen (wherein R represents lower alkyl or aryl, and the lower alkyl, the aryl, and the halogen have the same definitions as described above, respectively). Specific examples thereof include a 2′-modified nucleotide in which the 2′-OH is substituted with a substituent selected from the group consisting of a fluorine atom, methoxy, 2-(methoxy)ethoxy, 3-aminopropoxy, 2-[(N,N-dimethylamino)oxy]ethoxy, 3-(N, N-dimethylamino) propoxy, 2-[2-(N,N-dimethylamino)ethoxy]ethoxy, 2-(methylamino)-2-oxoethoxy, 2-(N-methylcarbamoyl) ethoxy, and 2-cyanoethoxy, and the like.

A phosphate-modified nucleotide may be any as long as it is a nucleotide in which a part or the whole of the chemical structure of a phosphate diester bond of the nucleotide is modified with an arbitrary substituent or is substituted with an arbitrary atom, and examples thereof include a nucleotide in which a phosphate diester bond is substituted with an alkyl phosphonate bond, and the like.

Compound (Ia) can also be obtained in the form of, for example, an acid addition salt, a metal salt, an ammonium salt, an organic amine addition salt, an amino acid addition salt, or the like.

Examples of the acid addition salt include inorganic acid salts such as hydrochlorides, sulfates, and phosphates; and organic acid salts such as acetates, maleates, fumarates, citrates, and methanesulfonates. Examples of the metal salt include alkali metal salts such as sodium salts and potassium salts; alkaline earth metal salts such as magnesium salts and calcium salts; aluminum salts; zinc salts; and the like. Examples of the ammonium salts include salts such as ammonium, and tetramethylammonium. Examples of the organic amine addition salts include addition salts such as morpholine, piperidine. Examples of the amino acid addition salts include addition salts such as lysine, glycine, phenylalanine.

Among Compounds (Ia), some may include geometric isomers, stereoisomers such as optical isomers, tautomers, and the like. All possible isomers including these, and mixtures thereof can be used in the present invention.

Further, Compounds (Ia) may sometimes exist in the form of an adduct with water or any of various solvents, and these adducts can also be used in the present invention.

Next, a method for producing the oligonucleotide of the present invention will be described.

A general synthetic method for an oligonucleotide includes, for example, an amidation step of nucleotides, an oligomerization step (including a deprotection step and the like), a duplexing step by annealing (as needed), and the like.

The oligonucleotide of the present invention can be produced by, for example, the following production method.

### (1) General Example of Oligomerization

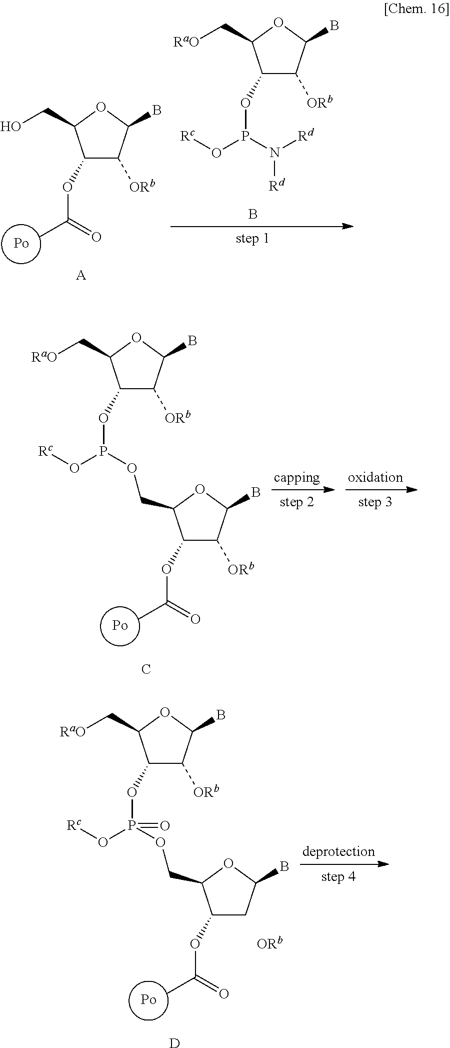

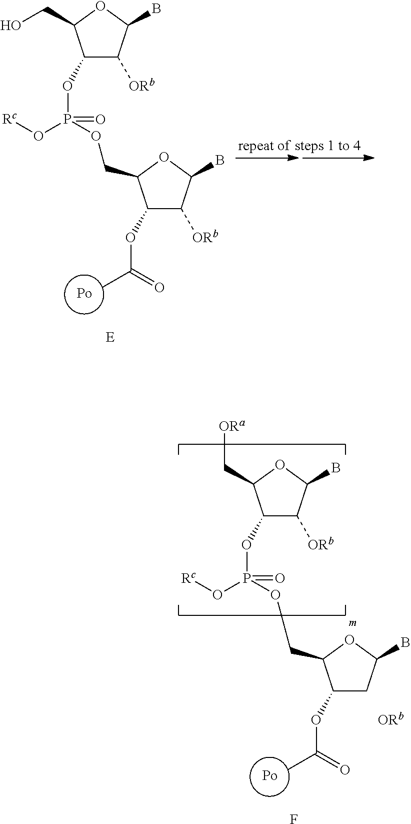

[In the formula, m represents an integer of, for example, 9 to 99; Po represents a solid-phase support of, for example, CPG (controlled pore glass) or the like; R^{a} represents a protective group which can be removed by a treatment with an acid, for example, trityl, p,p′-dimethoxytrityl, or the like; R^{b} represents a protective group which can be removed with a fluoride ion, for example, tert-butyldimethylsilyl or the like; R^{c} represents a protective group which can be removed by a treatment with a base, for example, 2-cyanoethyl or the like; R^{d} represents optionally substituted lower alkyl; and B represents a nucleobase (the nucleobase may be protected by one or more protective groups as needed, and in the case where the number of the protective groups is 2 or more, the respective protective groups may be the same or different). In the case where m is 2 or more, B's in number of m+1, R^{b}'s in number of m+1, and R^{c}'s in number of m may be the same or different, respectively, and R^{a}'s in the respective steps may be the same or different. Here, the lower alkyl has the same definition as described above, and the substituent of the optionally substituted lower alkyl has the same definition as the substituent of the optionally substituted lower alkyl described above.]

### Step 1

Compound C can be produced by reacting Compound A with Compound B in a solvent in the presence of a reaction accelerator at a temperature between 0° C. and 50° C. for 10 seconds to 30 minutes.

Examples of the solvent include dichloromethane, acetonitrile, toluene, ethyl acetate, tetrahydrofuran (THF), 1,4-dioxane, N,N-dimethylformamide (DMF), N-methylpyrrolidone (NMP), water, and the like. These can be used alone or as a mixture thereof.

Examples of the reaction accelerator include 1H-tetrazole, 4,5-dicyanoimidazole, 5-ethylthiotetrazole, 5-benzylthiotetrazole, and the like.

Compound A is obtained as, for example, a commercially available product.

Compound B can be produced by, for example, the following method.

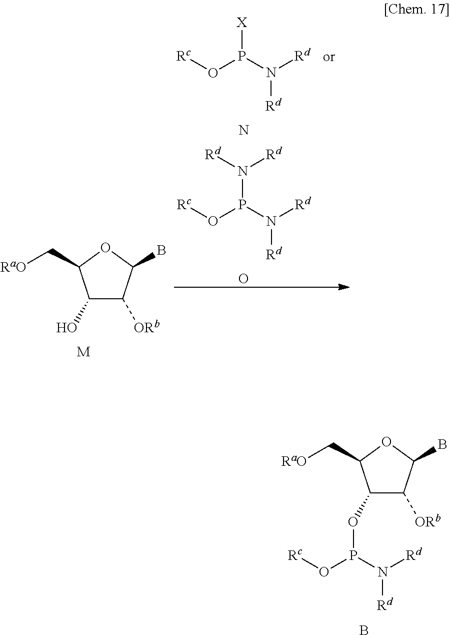

(In the formula, R^{a}, R^{b}, R^{c}, R^{d}, and B have the same definitions as described above, respectively, and X represents halogen. The halogen has the same definition as described above.)

Compound B can be produced by reacting Compound M with Compound N in a solvent in the presence of a base at a temperature between 0° C. and 100° C. for 10 seconds to 24 hours.

Examples of the solvent include dichloromethane, acetonitrile, toluene, ethyl acetate, THF, 1,4-dioxane, DMF, NMP, and the like. These can be used alone or as a mixture thereof.

Examples of the base include triethylamine, N,N-diisopropylethylamine, pyridine, and the like. These can be used alone or as a mixture thereof.

Further, Compound B can also be produced by reacting Compound M with Compound O in a solvent in the presence of a reaction accelerator at a temperature between 0° C. and 100° C. for 10 seconds to 24 hours.

Examples of the solvent include acetonitrile, THF, and the like. These can be used alone or as a mixture thereof.

Examples of the reaction accelerator include 1H-tetrazole, 4,5-dicyanoimidazole, 5-ethylthiotetrazole, 5-benzylthiotetrazole, and the like.

### Step 2

In Step 1, unreacted Compound A can be capped by reacting an acylation reagent with a base in a solvent at a temperature between 0° C. and 50° C. for 10 seconds to 30 minutes. At this time, the reaction can also be accelerated by adding a suitable additive.

Examples of the acylation reagent include acetic anhydride.

Examples of the solvent include dichloromethane, acetonitrile, ethyl acetate, THF, 1,4-dioxane, DMF, and the like. These can be used alone or as a mixture thereof.

Examples of the base include pyridine, 2,6-lutidine, and the like.

Examples of the additive include 4-dimethylaminopyridine, 1-methylimidazole, and the like.

### Step 3

Compound D can be produced by reacting Compound C with an oxidizing agent in a solvent in the presence of a base at a temperature between 0° C. and 50° C. for 10 seconds to 30 minutes.

Examples of the oxidizing agent include iodine, an aqueous hydrogen peroxide solution, m-chloroperoxybenzoic acid, peracetic acid, tert-butyl hydroperoxide, and the like. These can be used alone or as a mixture thereof.

Examples of the base include pyridine, 2,6-lutidine, and the like.

Examples of the solvent include dichloromethane, acetonitrile, ethyl acetate, THF, 1,4-dioxane, DMF, and the like. These can be used alone or as a mixture thereof.

### Step 4

Compound E can be produced by reacting Compound D with an acid in a solvent at a temperature between 0° C. and 50° C. for 10 seconds to 30 minutes.

Examples of the acid include dichloroacetic acid, trichloroacetic acid, trifluoroacetic acid, and the like.

Examples of the solvent include dichloromethane, chloroform, and the like.

Steps 1 to 4, and the following Steps 5 and 6 can also be performed by using a nucleic acid synthesizer.

### (2) General Example of Introduction of Nucleotide Residue (I) at 5′ End

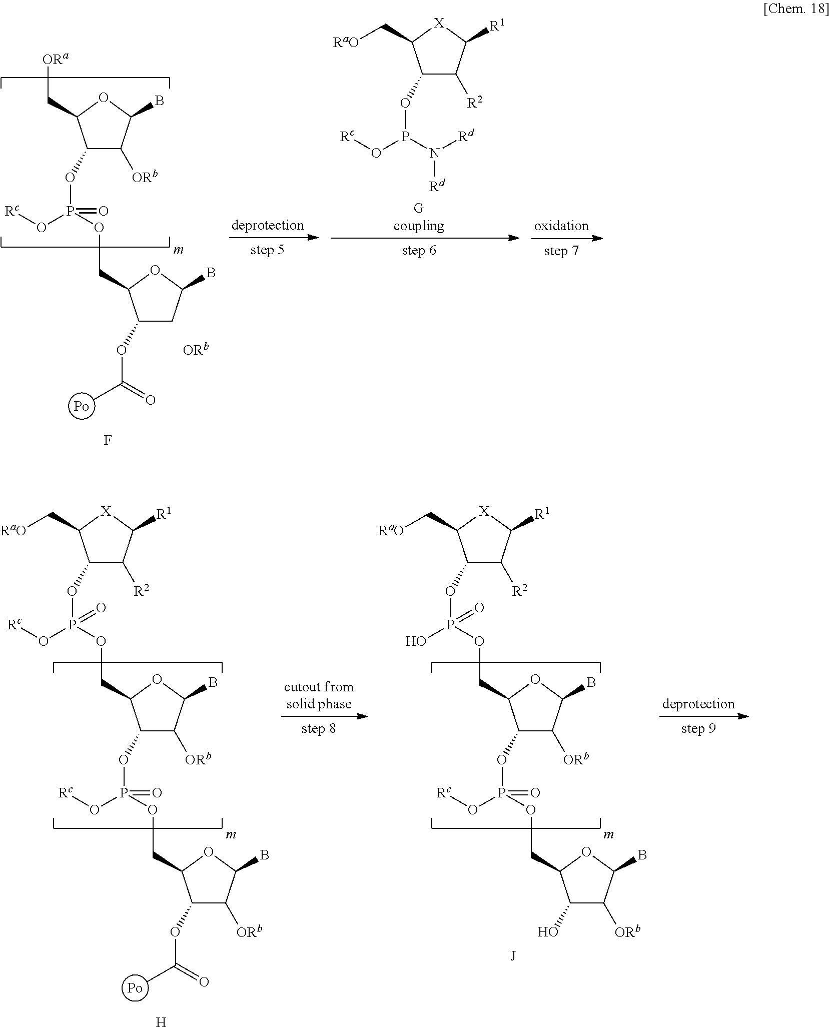

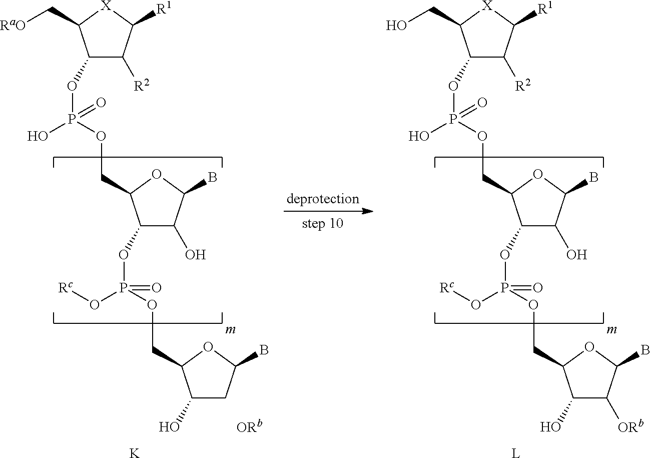

(In the formula, m, X, R¹, R², Po, R^{a}, R^{b}, R^{c}, and R^{d} have the same definitions as described above, respectively.)

### Step 5

Step 5 (deprotection of the protective group R^{a} of Compound F) can be performed in the same manner as the above-described Step 4.

### Step 6

Coupling of Compound F in which R^{a} is deprotected in Step 5 (hereinafter referred to as Compound Fa) with Compound G can be performed by, for example, the following method.

The production can be performed by reacting Compound Fa with 1 equivalent to a large excess amount of Compound G in a solvent in the presence of a reaction accelerator at a temperature between 0° C. and 50° C. for 10 seconds to 30 minutes.

Examples of the solvent include dichloromethane, acetonitrile, toluene, ethyl acetate, THF, 1,4-dioxane, DMF, NMP, water, and the like. These can be used alone or as a mixture thereof.

Examples of the reaction accelerator include 1H-tetrazole, 4,5-dicyanoimidazole, 5-ethylthiotetrazole, 5-benzylthiotetrazole, and the like.

Compound G is obtained as, for example, a commercially available product.

### Step 7

Compound H can be obtained in the same manner as in the above-described Step 3 (oxidation of a phosphorus atom).

### Step 8

By allowing a base to act on an oligonucleotide supported on a solid phase, the oligonucleotide can be cleaved from the solid phase. Namely, Compound J can be produced by treating Compound H with a base in a solvent at a temperature between −80° C. and 200° C. for 10 seconds to 72 hours.

Examples of the base include ammonia, methylamine, dimethylamine, ethylamine, diethylamine, 1,8-diazabicyclo[5.4.0]-7-undecene (DBU), and the like.

Examples of the solvent include water, methanol, ethanol, and the like.

Additionally, in this step, deprotection of the protective group for a nitrogen atom contained in B (nucleobase) is also performed simultaneously.

### Step 9

Compound K can be produced by reacting Compound J with a fluorinating reagent in a solvent at a temperature between −80° C. and 200° C. for 10 seconds to 72 hours. At this time, it is also possible to add a base to the reaction system.

Examples of the fluorinating reagent include hydrogen fluoride, triethylamine hydrofluoride, tetrabutylammonium fluoride (TBAF), and the like.

Examples of the base include triethylamine, N,N-diisopropylethylamine, and the like.

Examples of the solvent include dichloromethane, chloroform, acetonitrile, toluene, ethyl acetate, THF, 1,4-dioxane, DMF, N,N-dimethylacetamide (DMA), NMP, dimethyl sulfoxide (DMSO), and the like.

### Step 10

Compound L can be produced by treating Compound K with an acid in a solvent or in a column at a temperature between 0° C. and 50° C. for 5 minutes to 100 hours.

Examples of the acid include trifluoroacetic acid and the like.

Examples of the solvent include water, methanol, ethanol, acetonitrile, and the like. These can be used alone or as a mixture thereof.

Examples of the column include a C18 reverse-phase cartridge column and the like.

### (3) General Example of Production of Double-Stranded Oligonucleotide

After Compound L is reacted with an equimolar amount of a single-stranded oligonucleotide in a solvent at a temperature between 30° C. and 120° C. for 10 seconds to 72 hours, the reaction mixture is gradually cooled to room temperature over 10 minutes to 24 hours, whereby a double-stranded oligonucleotide can be produced.

Examples of the solvent include acetate buffer, Tris buffer, citrate buffer, phosphate buffer, water, and the like. These can be used alone or as a mixture thereof.

The single-stranded oligonucleotide to be reacted with Compound L is an oligonucleotide complementary to Compound L, but may contain one or more mismatched base pairs. Further, the base length thereof may be different.

In the above-described scheme, by variously changing nucleobases, the reaction conditions for the respective steps, and the like, a desired oligonucleotide can be obtained.

These can be performed according to the methods described in, for example,

(i) Tetrahedron, vol. 48 No. 12, pp. 2223-2311 (1992);

(ii) Current Protocols in Nucleic Acids Chemistry, John Wiley & Sons (2000);

(iii) Protocols for Oligonucleotides and Analogs, Human Press (1993);

(iv) Chemistry and Biology of Artificial Nucleic Acids, Wiley-VCH (2012);

(v) Genome Chemistry, Scientific Approach Using Artificial Nucleic Acids, Kodansha Ltd. (2003);

(vi) New Trends of Nucleic Acids Chemistry, Kagaku-Dojin Publishing Company, Inc. (2011); and the like.

### (4) General Method for Producing Nucleotide or Nucleoside Corresponding to Nucleotide Residue or Nucleoside Residue Represented by Formula (I)

Hereinafter, a general method for producing a nucleotide or a nucleoside corresponding to a nucleotide residue or a nucleoside residue represented by Formula (I) will be described, however, the method for producing a nucleotide residue or a nucleoside residue to be used in the present invention is not limited thereto.

In the production method described below, in the case where the defined group changes under the conditions for the production method or is not suitable for performing the production method, by using a method for introducing and removing a protective group conventionally used in organic synthesis chemistry (for example, a method described in Protective Groups in Organic Synthesis, fourth edition, written by T. W. Greene, John Wiley & Sons Inc. (2006), or the like) or the like, a target compound can be produced. Further, it is also possible to change the order of the reaction steps for introducing a substituent and the like as needed.

### Production Method 4-1

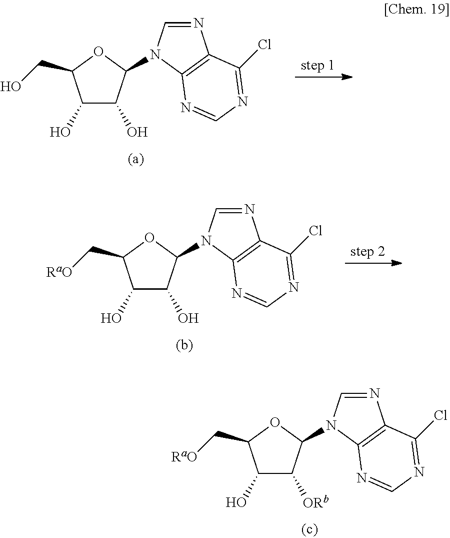

(In the formula, R^{a} and R^{b} have the same definitions as described above, respectively.)

### Step 1

Compound b can be produced by reacting Compound a with a corresponding alkylating agent in a solvent in the presence of a base at a temperature between 0° C. and 150° C. for 10 minutes to 3 days. The reaction can also be accelerated by a suitable activator.

Examples of the solvent include DMF, pyridine, dichloromethane, THF, ethyl acetate, 1,4-dioxane, NMP, and the like. These can be used alone or as a mixture thereof.

Examples of the base include pyridine, triethylamine, N-ethyl-N,N-diisopropylamine, 2,6-lutidine, and the like.

Examples of the alkylating agent include trityl chloride, p,p′-dimethoxytrityl chloride, and the like.

Examples of the activator include 4-dimethylaminopyridine and the like.

Compound a can be synthesized by, for example, a known method (a method described in Journal of Medicinal Chemistry, 2004, 47(6), 1987-1996).

### Step 2

Compound c can be produced by reacting Compound b with a silylating agent in a solvent in the presence of a silver salt and a base at a temperature between 0° C. and 80° C. for 10 minutes to 3 days.

Examples of the solvent include THF, ethylene glycol dimethyl ether (DME), and the like. These can be used alone or as a mixture thereof.

Examples of the silver salt include silver nitrate, silver perchlorate, and the like.

Examples of the base include triethylamine, 1,4-diazabicyclo[2,2,2]octane (DABCO), pyridine, and the like.

Examples of the silylating agent include tert-butyldimethylsilyl chloride and the like.

### Production Method 4-2

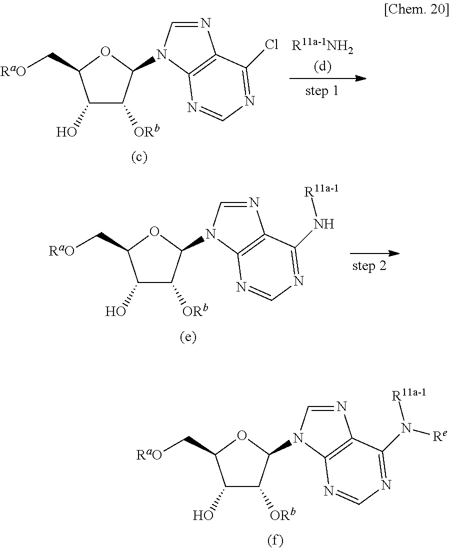

(In the formula, R^{a} and R^{b} have the same definitions as described above, respectively; R^{e} represents a protective group which can be removed with a base, for example, acetyl, benzoyl, or the like; and R^{11a-1} represents optionally substituted lower alkyl, optionally substituted lower alkenyl, optionally substituted lower alkynyl, optionally substituted aryl, optionally substituted aralkyl, optionally substituted aromatic heterocyclic alkyl, or an optionally substituted aromatic heterocyclic group in the definition of R^{11a} described above.)

### Step 1

Compound e can be produced by reacting Compound c with Compound d in a solvent or without solvent in the presence or absence of a base at a temperature between 0° C. and 150° C. for 1 hour to 1 week.

Examples of the solvent include DMF, pyridine, dichloromethane, THF, ethyl acetate, NMP, acetonitrile, and the like. These can be used alone or as a mixture thereof.

Examples of the base include triethylamine, N-ethyl-N,N-diisopropylamine, and the like.

### Step 2

Compound f can be produced by reacting Compound e with a silylating agent in a solvent at a temperature between 0° C. and 100° C. for 10 minutes to 3 hours, and then, by reacting the resulting product with an acylation agent at a temperature between 0° C. and 100° C. for 1 hour to 72 hours, and further treating the resulting product with water or an alcohol for 1 hour to 24 hours. The reaction can also be accelerated by allowing a suitable activator to coexist with the acylation agent.

Examples of the solvent include pyridine and the like.

Examples of the silylating agent include trimethylsilyl chloride, trifluoromethanesulfonyl trimethylsilyl, N,O-bis(trimethylsilyl)acetamide, 1,1,1,3,3,3-hexamethyldisilazane, and the like.

Examples of the acylation agent include acetic anhydride, acetyl chloride, benzoyl chloride, and the like.

Examples of the alcohol include methanol, ethanol, 1-propanol, and the like.

Examples of the activator include 4-dimethylaminopyridine.

### Production Method 4-3

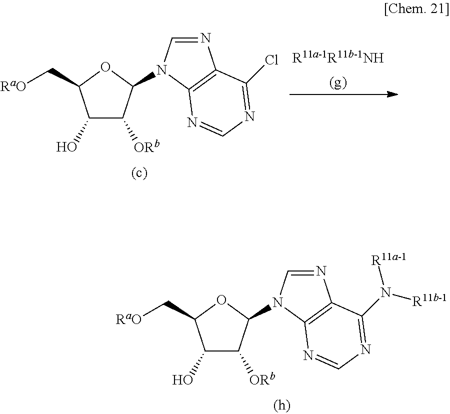

(In the formula, R^{a}, R^{b}, and R^{11a-1} have the same definitions as described above, respectively, and R^{11b-1} represents optionally substituted lower alkyl, optionally substituted lower alkenyl, optionally substituted lower alkynyl, optionally substituted aryl, optionally substituted aralkyl, optionally substituted aromatic heterocyclic alkyl, or an optionally substituted aromatic heterocyclic group in the definition of R^{11b} described above.)

Compound h can be produced by reacting Compound c with Compound g in a solvent or without solvent in the presence or absence of a base at a temperature between 0° C. and 150° C. for 1 hour to 1 week.

Examples of the solvent include DMF, pyridine, dichloromethane, THF, ethyl acetate, NMP, acetonitrile, and the like. These can be used alone or as a mixture thereof.

Examples of the base include triethylamine, N-ethyl-N,N-diisopropylamine, and the like.

### Production Method 4-4

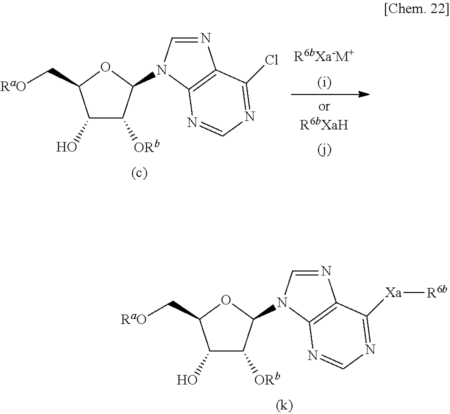

(In the formula, R^{a} and R^{b} have the same definitions as described above, respectively; R^{6b} represents optionally substituted lower alkyl, optionally substituted aryl, or an optionally substituted aromatic heterocyclic group; Xa represents an oxygen atom or a sulfur atom; and M represents an alkali metal atom. Here, the lower alkyl, the aryl, and the aromatic heterocyclic group have the same definitions as described above, respectively, the substituent of the optionally substituted lower alkyl has the same definition as the substituent of the optionally substituted lower alkyl described above, and the substituents of the optionally substituted aryl and the optionally substituted aromatic heterocyclic group have the same definitions as the substituents of the optionally substituted aryl and the optionally substituted aromatic heterocyclic group described above, respectively. The alkali metal atom represents a lithium atom, a sodium atom, or a potassium atom.)

Compound k can be produced by reacting Compound c with Compound i in a solvent at a temperature between 0° C. and 100° C. for 10 minutes to 3 days, or by reacting Compound c with Compound j in a solvent in the presence of a base at a temperature between 0° C. and 120° C. for 10 minutes to 3 days.

Examples of the solvent include methanol, ethanol, 2-propanol, THF, DME, DMF, NMP, and the like. These can be used alone or as a mixture thereof.

Examples of the base include sodium hydroxide, potassium hydroxide, sodium carbonate, potassium carbonate, sodium hydride, potassium hydride, tert-butoxy potassium, and the like.

### Production Method 4-5

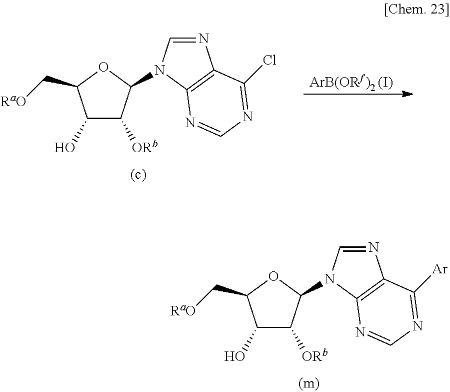

(In the formula, R^{a} and R^{b} have the same definitions as described above, respectively; Ar represents optionally substituted aryl or an optionally substituted aromatic heterocyclic group; and R^{f} represents a hydrogen atom or optionally substituted lower alkyl. Here, the lower alkyl, the aryl, and the aromatic heterocyclic group have the same definitions as described above, respectively, the substituent of the optionally substituted lower alkyl has the same definition as the substituent of the optionally substituted lower alkyl described above, and the substituents of the optionally substituted aryl and the optionally substituted aromatic heterocyclic group have the same definitions as the substituents of the optionally substituted aryl and the optionally substituted aromatic heterocyclic group described above, respectively.)

Compound m can be produced by reacting Compound c with Compound l in a solvent in the presence of a base and a palladium catalyst at a temperature between 0° C. and 120° C. for 30 minutes to 72 hours. The reaction can also be accelerated by adding a suitable phosphine to the reaction system.

Compound l is obtained as a commercially available product or can be prepared according to a known method (for example, Synthesis of Organic Compound VI, organic synthesis using metal, Jikken Kagaku Koza (Encyclopedia of Experimental Chemistry) 18, 5th Ed., p. 97, Maruzen (2005)) or a modified method thereof.

Examples of the base include potassium carbonate, potassium phosphate, potassium hydroxide, sodium hydroxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine)palladium, 1,1′-bis(diphenylphosphino)ferrocenedichloropalladium/dichloromethane (1:1) adduct, and the like.

Examples of the solvent include methanol, ethanol, dichloromethane, chloroform, 1,2-dichloroethane, toluene, ethyl acetate, acetonitrile, diethyl ether, THF, DME, dioxane, DMF, DMA, NMP, water, and the like. These can be used alone or as a mixture thereof.

Examples of the suitable phosphine include 1,2-bis(diphenylphosphino) ethane, 1,3-bis(diphenylphosphino)propane, 4,5′-bis(diphenylphosphino)-9,9′-dimethyxanthene (Xantphos), 2,2′-bis(diphenylphosphino)-1,1′-binaphthyl (BINAP), and the like.

### Production Method 4-6

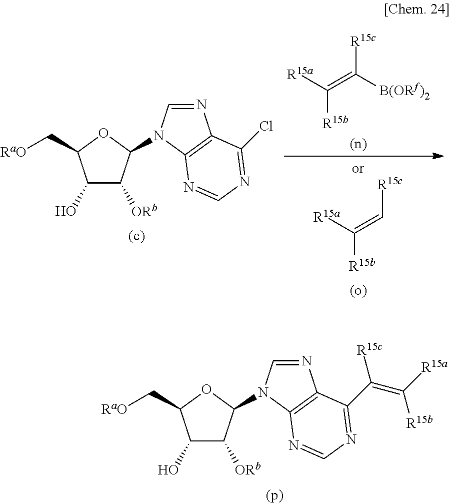

(In the formula, R^{a}, R^{b}, and R^{c} have the same definitions as described above, respectively; R^{15a}, R^{15b}, and R^{15c} each represent a hydrogen atom, optionally substituted lower alkyl, aryl, or an aromatic heterocyclic group. Here, the lower alkyl, the aryl, and the aromatic heterocyclic group have the same definitions as described above, respectively, and the substituent of the optionally substituted lower alkyl has the same definition as the substituent of the optionally substituted lower alkyl described above.)

Compound p can be produced by reacting Compound c with Compound n in a solvent in the presence of a base and a palladium catalyst at a temperature between 0° C. and 120° C. for 30 minutes to 72 hours. The reaction can also be accelerated by adding a suitable phosphine to the reaction system.

Compound n is obtained as a commercially available product.

Examples of the base include potassium carbonate, potassium phosphate, potassium hydroxide, sodium hydroxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine)palladium, 1,1′-bis(diphenylphosphino) ferrocenedichloropalladium/dichloromethane (1:1) adduct, and the like.

Examples of the solvent include methanol, ethanol, dichloromethane, chloroform, 1,2-dichloroethane, toluene, ethyl acetate, acetonitrile, diethyl ether, THF, DME, dioxane, DMF, DMA, NMP, water, and the like. These can be used alone or as a mixture thereof.

Further, Compound p can also be produced by reacting Compound c with Compound o in a solvent in the presence of a base and a palladium catalyst at a temperature between 0° C. and 140° C. for 30 minutes to 72 hours.

Examples of the base include potassium acetate, sodium hydrogen carbonate, potassium carbonate, potassium hydroxide, sodium hydroxide, sodium methoxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine)palladium, 1,1′-bis(diphenylphosphino)ferrocenedichloropalladium/dichloromethane (1:1) adduct, and the like.

Examples of the solvent include dichloromethane, chloroform, 1,2-dichloroethane, toluene, ethyl acetate, acetonitrile, diethyl ether, THF, DME, dioxane, DMF, DMA, NMP, and the like. These can be used alone or as a mixture thereof.

Examples of the suitable phosphine include 1,2-bis(diphenylphosphino)ethane, 1,3-bis(diphenylphosphino)propane, Xantphos, BINAP, and the like.

### Production Method 4-7

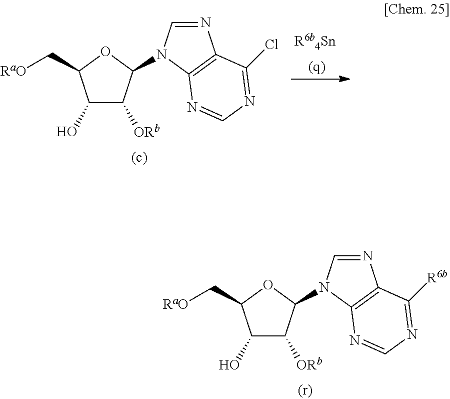

(In the formula, R^{a}, R^{b}, and R^{6b} have the same definitions as described above, respectively.)

Compound r can be produced by reacting Compound c with Compound q in a solvent in the presence of a palladium catalyst at a temperature between 0° C. and 150° C. The reaction can also be accelerated by adding a suitable additive and/or a suitable phosphine to the reaction system.

Examples of the solvent include methanol, ethanol, dichloromethane, chloroform, 1,2-dichloroethane, toluene, ethyl acetate, acetonitrile, diethyl ether, THF, DME, dioxane, DMF, DMA, NMP, and the like. These can be used alone or as a mixture thereof.

Compound q is obtained as a commercially available product.

Examples of the palladium catalyst include palladium acetate, tris(dibenzylideneacetone)dipalladium, tetrakis(triphenylphosphine)palladium, 1,1′-bis(diphenylphosphino) ferrocenedichloropalladium/dichloromethane (1:1) adduct, and the like.

Examples of the suitable additive include lithium chloride, cesium fluoride, and the like.

Examples of the suitable phosphine include 1,2-bis(diphenylphosphino)ethane, 1,3-bis(diphenylphosphino)propane, Xantphos, BINAP, and the like.

### Production Method 4-8

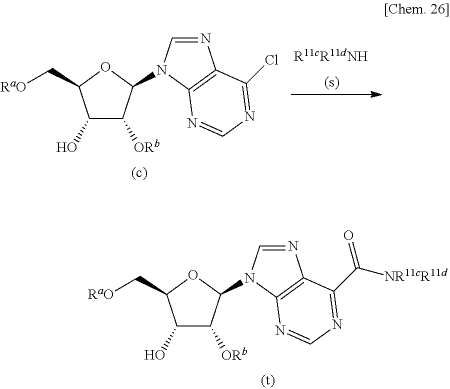

(In the formula, R^{a}, R^{b}, R^{11c}, and R^{11d} have the same definitions as described above, respectively.)

Compound t can be produced by reacting Compound c with Compound s in a solvent in a carbon monoxide atmosphere in the presence of a base and a palladium catalyst at a temperature between room temperature and 120° C. for 1 hour to 72 hours. The reaction can also be accelerated by adding a suitable phosphine to the reaction system.

Examples of the solvent include dichloromethane, chloroform, 1,2-dichloroethane, toluene, ethyl acetate, acetonitrile, diethyl ether, THF, DME, dioxane, DMF, DMA, NMP, and the like. These can be used alone or as a mixture thereof.

Examples of the base include sodium acetate, potassium acetate, sodium hydrogen carbonate, potassium carbonate, sodium methoxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine)palladium, 1,1′-bis(diphenyphosphino)ferrocenedichloropalladium/dichloromethane (1:1) adduct, and the like.

Examples of the phosphine include 1,2-bis(diphenylphosphino)ethane, 1,3-bis(diphenylphosphino)propane, Xantphos, BINAP, and the like.

### Production Method 4-9

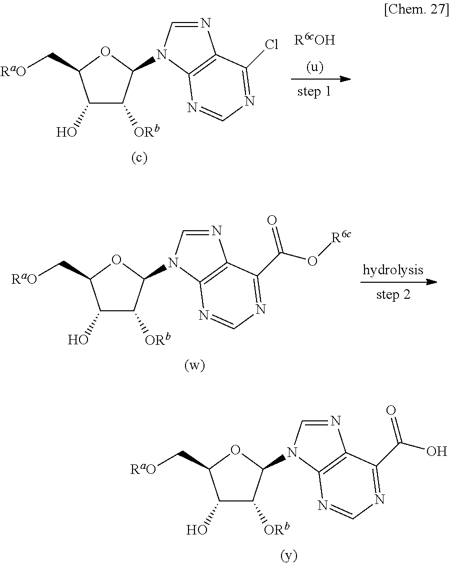

(In the formula, R^{a} and R^{b} have the same definitions as described above, respectively; and R^{6c} represents optionally substituted lower alkyl, optionally substituted aryl, or an optionally substituted aromatic heterocyclic group. Here, the lower alkyl, the aryl, and the aromatic heterocyclic group have the same definitions as described above, respectively, the substituent of the optionally substituted lower alkyl has the same definition as the substituent of the optionally substituted lower alkyl described above, and the substituents of the optionally substituted aryl and the optionally substituted aromatic heterocyclic group have the same definitions as the substituents of the optionally substituted aryl and the optionally substituted aromatic heterocyclic group described above, respectively.)

### Step 1

Compound w can be produced by reacting Compound c with Compound u in a solvent in a carbon monoxide atmosphere in the presence of a base and a palladium catalyst at a temperature between room temperature and 120° C. for 1 hour to 72 hours. The reaction can also be accelerated by adding a suitable phosphine to the reaction system.

Examples of the solvent include methanol, ethanol, 1-propanol, 2-propanol, 1-butanol, tert-butyl alcohol, dichloromethane, chloroform, 1,2-dichloroethane, toluene, ethyl acetate, acetonitrile, diethyl ether, THF, DME, dioxane, DMF, DMA, NMP, and the like. These can be used alone or as a mixture thereof.

Examples of the base include sodium acetate, potassium acetate, sodium hydrogen carbonate, potassium carbonate, sodium methoxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine)palladium, 1,1′-bis(diphenylphosphino)ferrocenedichloropalladium/dichloromethane (1:1) adduct, and the like.

Examples of the phosphine include 1,2-bis(diphenylphosphino)ethane, 1,3-bis(diphenylphosphino)propane, Xantphos, BINAP, and the like.

### Step 2

Compound y can be produced by treating Compound w in a solvent in the presence of a base at a temperature between 0° C. and 100° C. for 5 minutes to 72 hours.

Examples of the base include potassium carbonate, lithium hydroxide, potassium hydroxide, sodium hydroxide, sodium methoxide, and the like.

Examples of the solvent include a solvent containing water, and examples of the solvent include methanol, ethanol, dichloromethane, chloroform, 1,2-dichloroethane, toluene, ethyl acetate, acetonitrile, diethyl ether, THF, DME, dioxane, DMF, DMA, NMP, pyridine, and the like. These are used by mixing with water or by mixing with one another and then adding water thereto.

### Production Method 4-10

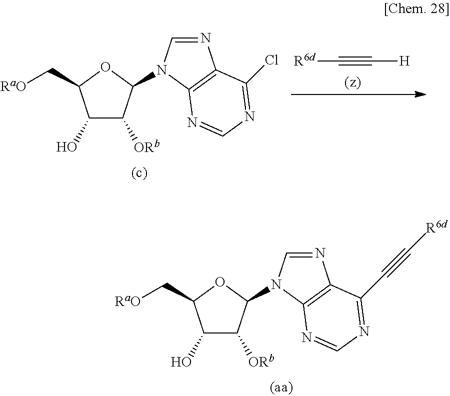

(In the formula, R^{a} and R^{b} have the same definitions as described above, respectively; and R^{6d} represents optionally substituted lower alkyl, aryl, or an aromatic heterocyclic group. Here, the lower alkyl, the aryl, and the aromatic heterocyclic group have the same definitions as described above, respectively, and the substituent of the optionally substituted lower alkyl has the same definition as the substituent of the optionally substituted lower alkyl described above.)

Compound aa can be produced by reacting Compound c with Compound z in a solvent in the presence of a copper salt, a base, and a palladium catalyst at a temperature between room temperature and 150° C. for 1 hour to 72 hours. The reaction can also be accelerated by adding a suitable phosphine to the reaction system.

Examples of the solvent include dichloromethane, chloroform, 1,2-dichloroethane, toluene, ethyl acetate, acetonitrile, diethyl ether, THF, DME, dioxane, DMF, DMA, NMP, and the like. These can be used alone or as a mixture thereof.

Examples of the copper salt include copper(I) iodide and the like.

Examples of the base include sodium acetate, potassium acetate, sodium hydrogen carbonate, potassium carbonate, sodium methoxide, potassium tert-butoxide, triethylamine, diisopropylethylamine, N-methylmorpholine, pyridine, DBU, and the like.

Examples of the palladium catalyst include palladium acetate, tris(dibenzylideneacetone)dipalladium, tetrakis(triphenylphosphine) palladium, 1,1′-bis(diphenylphosphino)ferrocenedichloropalladium/dichloromethane (1:1) adduct, and the like.

Examples of the phosphine include 1,2-bis(diphenylphosphino)ethane, 1,3-bis(diphenylphosphino)propane, Xantphos, BINAP, and the like.

### Production Method 4-11

By using Compound ae obtained by the following method, a nucleoside to be used as a starting material for the production of the oligonucleotide of the present invention can be obtained according to the above-described production methods 4-2 to 4-10.

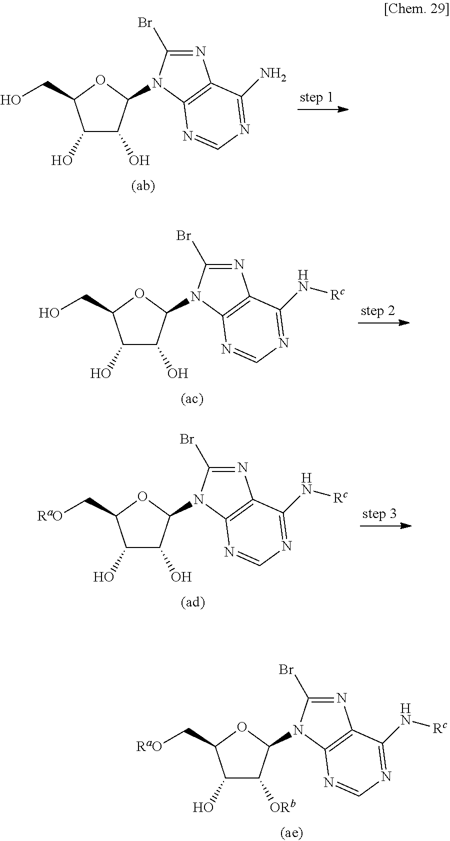

(In the formula, R^{a}, R^{b}, and R^{c} have the same definitions as described above, respectively.)

### Step 1

Compound ac can be produced by reacting Compound ab with a silylating agent in a solvent at a temperature between 0° C. and 100° C. for 10 minutes to 3 hours, and then, by reacting the resulting product with an acylation agent at a temperature between 0° C. and 100° C. for 1 hour to 72 hours, and further treating the resulting product with water or an alcohol for 1 hour to 24 hours.

Examples of the solvent include pyridine and the like.

Examples of the silylating agent include trimethylsilyl chloride, trifluoromethanesulfonyl trimethylsilyl, N,O-bis(trimethylsilyl)acetamide, 1,1,1,3,3,3-hexamethyldisilazane, and the like.

Examples of the acylation agent include acetic anhydride, acetyl chloride, benzoyl chloride, and the like.

Examples of the alcohol include methanol, ethanol, 1-propanol, and the like.

Compound ab can be synthesized by, for example a known method (a method described in Tetrahedron, 1970, 26, 4251-4259].

### Step 2

Compound ad can be produced by reacting Compound ac with a corresponding alkylating agent in a solvent in the presence of a base at a temperature between 0° C. and 150° C. for 10 minutes to 3 days. The reaction can also be accelerated by a suitable activator.

Examples of the solvent include DMF, pyridine, dichloromethane, THF, ethyl acetate, 1,4-dioxane, NMP, and the like. These can be used alone or as a mixture thereof.

Examples of the base include pyridine, triethylamine, N-ethyl-N,N-diisopropylamine, 2,6-lutidine, and the like.

Examples of the alkylating agent include trityl chloride, p,p′-dimethoxytrityl chloride, and the like.

Examples of the activator include 4-dimethylaminopyridine and the like.

### Step 3

Compound ae can be produced by reacting Compound ad with a silylating agent in a solvent in the presence of a silver salt and a base at a temperature between 0° C. and 80° C. for 10 minutes to 3 days.

Examples of the solvent include THF, DME, and the like. These can be used alone or as a mixture thereof.

Examples of the silver salt include silver nitrate, silver perchlorate, and the like.

Examples of the base include triethylamine, DABCO, pyridine, and the like.

Examples of the silylating agent include tert-butyldimethylsilyl chloride and the like.

### Production Method 4-12

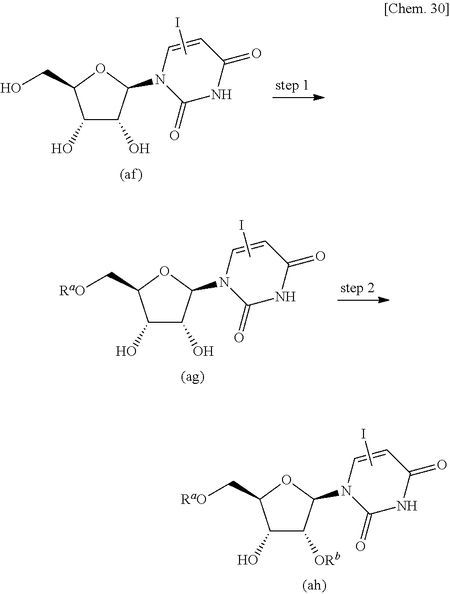

(In the formula, R^{a} and R^{b} have the same definitions as described above, respectively.)

### Step 1

Compound ag can be produced by reacting Compound af with a corresponding alkylating agent in a solvent in the presence of a base at a temperature between 0° C. and 150° C. for 10 minutes to 3 days. The reaction can also be accelerated by a suitable activator.

Examples of the solvent include DMF, pyridine, dichloromethane, THF, ethyl acetate, 1,4-dioxane, NMP, and the like. These can be used alone or as a mixture thereof.

Examples of the base include pyridine, triethylamine, N-ethyl-N,N-diisopropylamine, 2,6-lutidine, and the like.

Examples of the alkylating agent include trityl chloride, p,p′-dimethoxytrityl chloride, and the like.

Examples of the activator include 4-dimethylaminopyridine and the like.

Compound af can be synthesized by, for example, a known method (a method described in Journal of Medicinal Chemistry, 2004, 50(5), 915-921 and WO2011/51733).

### Step 2

Compound ah can be produced by reacting Compound ag with a silylating agent in a solvent in the presence of a silver salt and a base at a temperature between 0° C. and 80° C. for 10 minutes to 3 days.

Examples of the solvent include THF, DME, and the like. These can be used alone or as a mixture thereof.

Examples of the silver salt include silver nitrate, silver perchlorate, and the like.

Examples of the base include triethylamine, DABCO, pyridine, and the like.

Examples of the silylating agent include tert-butyldimethylsilyl chloride and the like.

### Production Method 4-13

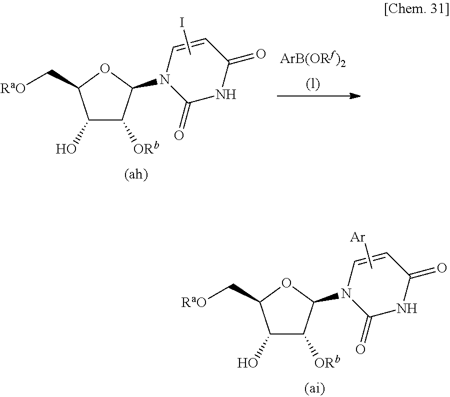

(In the formula, R^{a}, R^{b}, R^{f}, and Ar have the same definitions as described above, respectively.)

Compound ai can be produced according to the production method 4-5 using Compound ah.

### Production Method 4-14

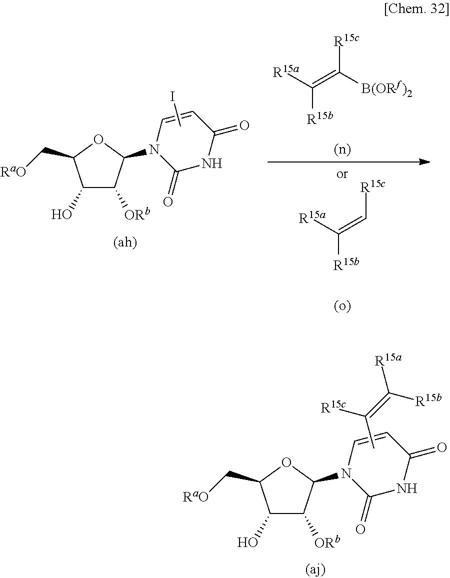

(In the formula, R^{a}, R^{b}, R^{f}, R^{15a}, R^{15b}, and R^{15c} have the same definitions as described above, respectively.)

Compound aj can be produced according to the production method 4-6 using Compound ah.

### Production Method 4-15

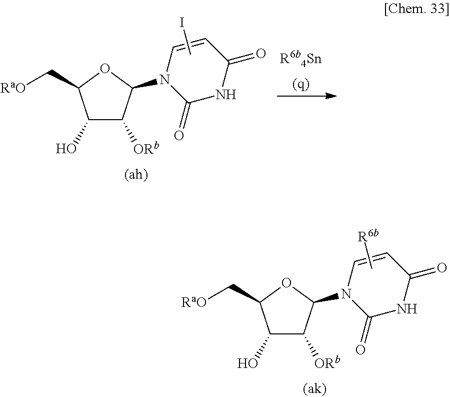

(In the formula, R^{a}, R^{b}, and R^{6b} have the same definitions as described above, respectively.)

Compound ak can be produced according to the production method 4-7 using Compound ah.

### Production Method 4-16

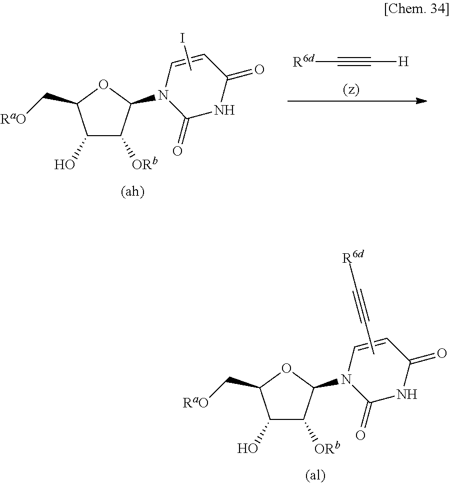

(In the formula, R^{a}, R^{b}, and R^{6d} have the same definitions as described above, respectively.)

Compound al can be produced according to the production method 4-10 using Compound ah.

The transformation of each group in the compounds included in the above-described respective production methods can also be performed by a known method (for example, a method described in Comprehensive Organic Transformations 2nd edition, written by R. C. Larock, Vch Verlagsgesellscaft Mbh, 1999, or the like) or a modified method thereof.

A desired nucleotide or nucleoside can be obtained by performing deprotection and phosphorylation of a hydroxy group of the product obtained through each of the above-described respective production methods according to a known method (for example, a method described in Journal of Medicinal Chemistry, vol. 55, pp. 1478-1489, 2012, or the like).

The intermediates and the desired compounds in the above-described respective production methods can be isolated and purified through a separation and purification method conventionally used in organic synthesis chemistry, for example, filtration, extraction, washing, drying, concentration, recrystallization, various types of chromatography, or the like. Further, the intermediate can also be subjected to the subsequent reaction without particularly undergoing purification.

The nucleotide or nucleoside can also be obtained in the form of a salt such as an acid addition salt, a metal salt, an ammonium salt, an organic amine addition salt, or an amino acid addition salt.

Examples of the acid addition salt include inorganic acid salts such as hydrochlorides, sulfates, and phosphates; organic acid salts such as acetates, maleates, fumarates, citrates, and methanesulfonates. Examples of the metal salt include alkali metal salts such as sodium salts and potassium salts; alkaline earth metal salts such as magnesium salts and calcium salts; aluminum salts; zinc salts; and the like. Examples of the ammonium salts include salts such as ammonium and tetramethylammonium. Examples of the organic amine addition salts include addition salts such as morpholine, and piperidine. Examples of the amino acid addition salts include addition salts such as lysine, glycine, and phenylalanine.

To obtain a salt of the nucleotide or the nucleoside, when the nucleotide or the nucleoside is obtained in the form of a salt, it may be purified as it is. Further, when the nucleotide or the nucleoside is obtained in a free form, the nucleotide or the nucleoside may be dissolved or suspended in a suitable solvent, followed by addition of an acid or a base to form a salt. Then, the resulting salt may be isolated and purified.

Among the nucleotides or the nucleosides, some may include geometric isomers, stereoisomers such as optical isomers, tautomers, and the like. All possible isomers including these and mixtures thereof can be used in the present invention.

Further, the nucleotides or the nucleosides may sometimes exist in the form of an adduct with water or any of various solvents, and these adducts can also be used in the present invention.

Specific examples of the oligonucleotide of the present invention are shown in Tables 1 and 2. In Tables 1 and 2, X included in the sequences of the antisense strands means 8-bromo-2′-deoxyadenosine monophosphate.

TABLE 1

| | sense strand | | antisense strand | |
|---|---|---|---|---|
| oligo- | sequence | sequence | sequence | sequence |
| nucleotide | (5′→3′) | number | (5′→3′) | number |
| 239-BrdA | UGCAGCGAGAAUA | 1 | XCAAGCUAUUC | 2 |
| | GCUUGUAG | | UCGCUGCACA | |
| 874-BrdA | UAGCUUCUUCGCU | 3 | XCUCUUAGCGA | 4 |
| | AAGAGUAC | | AGAAGCUAAA | |
| 904-BrdA | CAAGUACGACCUA | 5 | XUUGCUUAGGU | 6 |
| | AGCAAUUU | | CGUACUUGUC | |
| 1084-BrdA | AGGCAAGGUGGUG | 7 | XAAGGGCACCA | 8 |
| | CCCUUUUU | | CCUUGCCUAC | |
| 1203-BrdA | UUAACAACCCCGA | 9 | XUAGCCUCGGG | 10 |
| | GGCUAUAA | | GUUGUUAACG | |
| 1556-BrdA | GACGAGGUGCCUA | 11 | XUCCUUUAGGC | 12 |
| | AAGGAUUG | | ACCUCGUCCA | |

TABLE 2

| | sense strand | | antisense strand | |
|---|---|---|---|---|
| oligo- | sequence | sequence | sequence | Sequence |
| nucleotide | (5′→3′) | number | (5′→3′) | number |
| 217-BrdA | GCGCCUGGUCACC | 13 | XGCCCUGGUGA | 14 |
| | AGGGCUGC | | CCAGGCGCCC | |
| 278-BrdA | CCCUUCAUUGACC | 15 | XGUUGAGGUCA | 16 |
| | UCAACUAC | | AUGAAGGGGU | |
| 516-BrdA | GAGCCAAAAGGGU | 17 | XUGAUGACCCU | 18 |
| | CAUCAUCU | | UUUGGCUCCC | |
| 624-BrdA | CCUGCACCACCAA | 19 | XAGCAGUUGGU | 20 |
| | CUGCUUAG | | GGUGCAGGAG | |
| 715-BrdA | CACUGCCACCCAG | 21 | XGUCUUCUGGG | 22 |
| | AAGACUGU | | UGGCAGUGAU | |
| 816-BrdA | AGGCUGUGGGCAA | 23 | XUGACCUUGCC | 24 |
| | GGUCAUCC | | CACAGCCUUG | |
| 936-BrdA | AUGAUGACAUCAA | 25 | XCCUUCUUGAU | 26 |
| | GAAGGUGG | | GUCAUCAUAU | |
| 1096-BrdA | CAAGCUCAUUUCC | 27 | XUACCAGGAAA | 28 |
| | UGGUAUGA | | UGAGCUUGAC | |
| 1134-BrdA | GCAACAGGGUGGU | 29 | XGGUCCACCAC | 30 |
| | GGACCUCA | | CCUGUUGCUG | |

The oligonucleotide of the present invention can be introduced into a cell by using a carrier for use in transfection, preferably a cationic carrier such as a cationic liposome. Further, it can also be directly introduced into a cell by a calcium phosphate method, an electroporation method, a microinjection method, or the like.

Next, the activity of the oligonucleotide of the present invention will be specifically described by showing Test Examples.

### Test Example 1

### RNAi Activity of Luciferase-Targeting siRNA

The RNAi activity of a luciferase-targeting siRNA having 8-bromo-2′-deoxyadenosine monophosphate at the 5′ end of the antisense strand was evaluated by using the level of inhibition of luciferase luminescence as an index as described below.

In a culture dish (Assay plate, 96-well, with Lid, Cat. No. 3917, manufactured by Costar Co., Ltd.), human cervical cancer-derived cell line Hela cells (CCL-2, purchased from ATCC) transfected with a luciferase expression vector (pGL4.50 [luc2/CMV/Hygro] Vector, Promega Corporation) were suspended in RPMI medium (Invitrogen, 11875093) containing 10% fetal bovine serum, and the resulting cell suspension was inoculated into each well at 50 μL/well to give 5000 cells/well.

An siRNA was diluted with OPTI-MEM (Invitrogen, 31985-070). Lipofectamine RNAiMAX (Invitrogen, 13778-075) was diluted with OPTI-MEM. The thus prepared dilutions were mixed with each other to form an siRNA-lipofectamine RNAiMAX complex. By adding a solution of the thus prepared siRNA-Lipofectamine RNAiMAX (Invitrogen, 13778-075) complex was added to each well containing the cell suspension at 10 μL/well, the siRNA was introduced into the Hela cells. The final concentration of the siRNA was set to one value: 100 pmol/L, or the following four values: 3.2 pmol/L, 16 pmol/L, 80 pmol/L, and 400 pmol/L, and N was set to 6. Further, as a negative control group, cells to which only Lipofectamine RNAiMAX was added were inoculated. As the siRNA, 239-BrdA, 874-BrdA, 904-BrdA, 1084-BrdA, 1203-BrdA, and 1556-BrdA shown in Table 1 were used, and for comparison, a test was performed in the same manner also for siRNAs having adenosine monophosphate at a position corresponding to that of 8-bromo-2′-deoxyadenosine monophosphate of each siRNA (Table 3). Further, a test was performed in the same manner also for siRNAs having adenosine, guanosine, cytidine, or uridine at a position corresponding to that of 8-bromo-2′-deoxyadenosine monophosphate of 874-BrdA (Table 4).

The cells after introduction of the siRNA were cultured under the conditions of 37° C. and 5% CO₂ for 24 hours.

For the cells after culture, Steady-Glo Luciferase Assay System (Promega E2520), which is a commercially available luciferase assay reagent, was added to each well at 40 μL/well according to the accompanying protocol. After the cells were incubated for 10 minutes, the amount of luminescence (cps) per second in each well was measured using ARVO (PerkinElmer) according to the protocol.

Simultaneously with the measurement of the amount of luminescence in the luciferase-targeting siRNA treated group, the measurement of the amount of luminescence in the negative control group was performed, and the RNAi effect on each of the siRNA-introduced samples was expressed as a relative ratio when the amount of luminescence in the group in which the siRNA was not introduced (negative control group) was taken as 1.

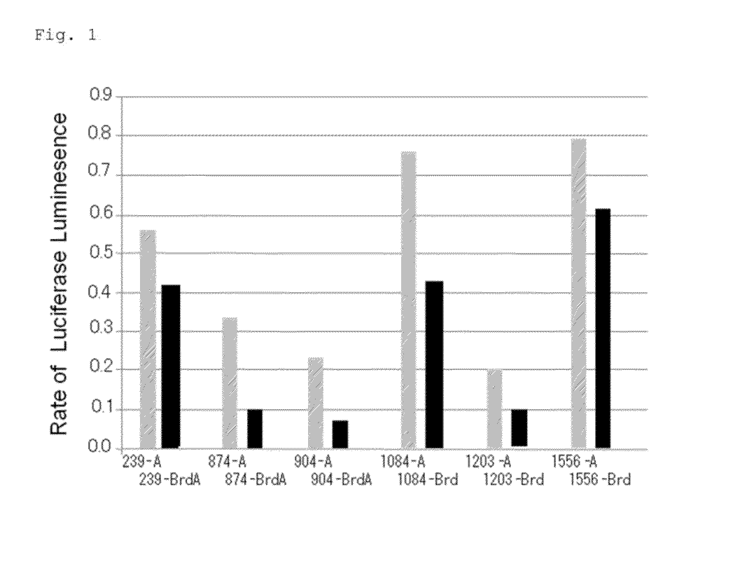

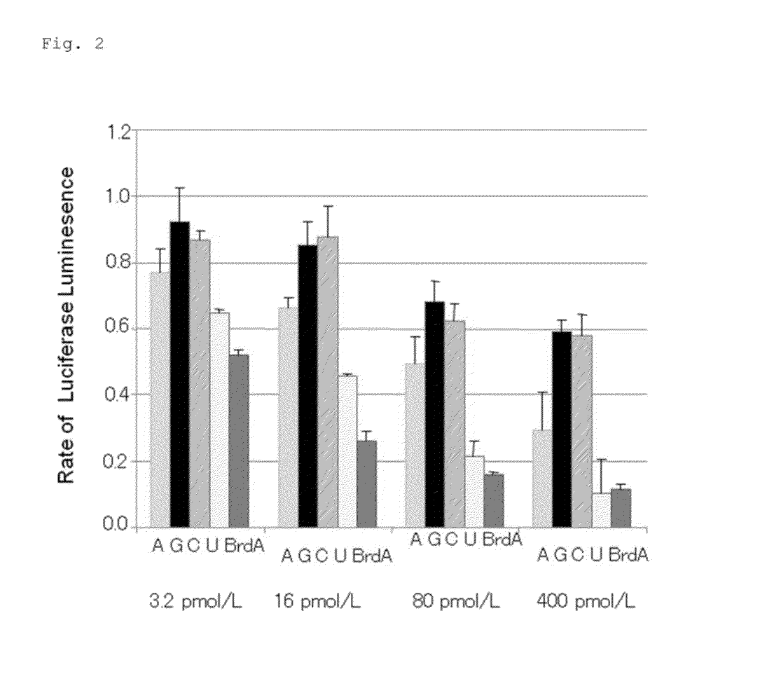

The results of this test are shown in FIGS. 1 and 2. FIG. 1 is a view showing comparison between the siRNA of the present invention and the siRNA in which 8-bromo-2′-deoxyadenosine monophosphate in the sequence of the siRNA of the present invention was replaced with adenosine monophosphate (Table 3) at a final concentration of 100 pmol/L. FIG. 2 is a view showing comparison between 874-BrdA, which is the siRNA of the present invention, and the siRNA in which 8-bromo-2′-deoxyadenosine monophosphate in the sequence of the 874-BrdA was replaced with adenosine, guanosine, cytidine, or uridine (Table 4) at final concentrations of 3.2 pmol/L, 16 pmol/L, 80 pmol/L, and 400 pmol/L (in FIGS. 1 and 2, the vertical axis represents the ratio of luminescence when the amount of luminescence in the negative control group was taken as 1). Additionally, by the Kruskal-Wallis test, it was determined whether or not there is a significant difference. In the statistical analysis, statistical analysis software SAS (Release 9.2, SAS Institute Inc.) was used. In Table 5, the results of the significant difference test for comparison between the ratio of inhibition of luminescence by 239-BrdA, 874-BrdA, 904-BrdA, 1084-BrdA, 1203-BrdA, or 1556-BrdA and the ratio of inhibition of luminescence by 239-A, 874-A, 904-A, 1084-A, 1203-A, or 1556-A are shown. A p-value of 0.05 or less was obtained in all the cases, and it is found that the ratio of inhibition of luminescence was significantly improved in the case of the siRNA in which adenosine monophosphate was replaced with 8-bromo-2′-deoxyadenosine monophosphate.

TABLE 3

| | sense strand | | antisense strand | |
|---|---|---|---|---|
| oligo- | sequence | sequence | sequence | sequence |
| nucleotide | (5′→3′) | number | (5′→3′) | number |
| 239-BrdA | UGCAGCGAGAAUA | 1 | XCAAGCUAUUC | 2 |
| | GCUUGUAG | | UCGCUGCACA | |
| 239-A | UGCAGCCGAAUAG | 31 | ACAAGCUAUUC | 32 |
| | CUUGUAG | | UCGCUGCACA | |
| 874-BrdA | UAGCUUCUUCGCU | 3 | XCUCUUAGCGA | 4 |
| | AAGAGUAC | | AGAAGCUAAA | |
| 874-A | UAGCUUCUUCGCU | 33 | ACUCUUAGCGA | 34 |
| | AAGAGUAC | | AGAAGCUAAA | |
| 904-BrdA | CAAGUACGACCUA | 5 | XUUGCUUAGGU | 6 |
| | AGCAAUUU | | CGUACUUGUC | |
| 904-A | CAAGUACGACCUA | 35 | AUUGCUUAGGU | 36 |
| | AGCAAUUU | | CGUACUUGCU | |
| 1084-BrdA | AGGCAAGGUGGUG | 7 | XAAGGGCACCA | 8 |
| | CCCUUUUU | | CCUUGCCUAC | |
| 1084-A | AGGCAAGGUGGUG | 37 | AAAGGGCACCA | 38 |
| | CCCUUUUU | | CCUUGCCUAC | |
| 1203-BrdA | UUAACAACCCCGA | 9 | XUAGCCUCGGG | 10 |
| | GGCUAUAA | | GUUGUUAACG | |
| 1203-A | UUAACAACCCCGA | 39 | AUAGCCUCGGG | 40 |
| | GGCUAUAA | | GUUGUUAACG | |
| 1556-BrdA | GACGAGGUGCCUA | 11 | XUCCUUUAGGC | 12 |
| | AAGGAUUG | | ACCUCGUCCA | |
| 1556-A | GACGAGGUGCCUA | 41 | AUCCUUUAGGC | 42 |
| | AAGGAUUG | | ACCUCGUCCA | |

TABLE 4

| | Sense strand | | antisense strand | |
|---|---|---|---|---|
| oligo- | sequence | sequence | sequence | Sequence |
| nucleotide | (5′→3′) | number | (5′→3′) | number |
| 874-BrdA | UAGCUUCUUCGCU | 3 | XCUCUUAGCGA | 4 |
| | AAGAGUAC | | AGAAGCUAAA | |
| 874-A | UAGCUUCUUCGCU | 33 | ACUCUUAGCGA | 34 |
| | AAGAGUAC | | AGAAGCUAAA | |
| 874-G | UAGCUUCUUCGCU | 43 | GCUCUUAGCGA | 44 |
| | AAGAGCAC | | AGAAGCUAAA | |
| 874-C | UAGCUUCUUCGCU | 45 | CCUCUUAGCGA | 46 |
| | AAGAGGAC | | AGAAGCUAAA | |
| 874-U | UAGCUUCUUCGCU | 47 | UCUCUUAGCGA | 48 |
| | AAGAGAAC | | AGAAGCUAAA | |

TABLE 5

| | 239- | 874- | 904- | 1084- | 1203- | 1556- |
|---|---|---|---|---|---|---|
| siRNA | BrdA | BrdA | BrdA | BrdA | BrdA | BrdA |
| p | 0.01 | 0.004 | 0.004 | 0.01 | 0.02 | 0.04 |

### Test Example 2

### RNAi Activity of Luciferase-Targeting siRNA

The RNAi activity of a luciferase-targeting siRNA having 8-bromo-2′-deoxyadenosine monophosphate at the 5′ end of the antisense strand was evaluated by measuring the inhibitory effect on the expression of the mRNA of luciferase (GL4) as described below.

In a culture dish (Multidish 24 wells, Cat. No. 142475, manufactured by Nunc, Inc.), human cervical cancer-derived cell line Hela cells (CCL-2, purchased from ATCC) were suspended in RPMI medium (Invitrogen, 11875093) containing 10% fetal bovine serum, and the resulting cell suspension was inoculated into each well at 500 μL/well to give 50000 cells/well. Thereto, a solution of an siRNA-Lipofectamine RNAiMAX (Invitrogen, 13778-075) complex mixed in OPTI-MEM (Invitrogen, 31985-070) was added at 100 μL/well, whereby the siRNA was introduced into the Hela cells. The final concentration of the siRNA was set to the following seven values: 10000 pmol/L, 2000 pmol/L, 400 pmol/L, 80 pmol/L, 16 pmol/L, 3.2 pmol/L, and 0.64 pmol/L. Further, as a negative control group, cells to which only RNAi MAX was added were inoculated.

The cells after introduction of the siRNA were cultured under the conditions of 37° C. and 5% CO₂ for 24 hours. In order to collect RNA, an RNA extraction kit (RNeasy 74106) of Qiagen, Inc. was used. The cells after culture were washed once with a phosphate buffer, and then lysed with RLT buffer (attached to RNeasy) and collected. Then, the total RNA was collected according to the protocol attached to the kit. By using the total RNA (1 μg) as a template, a reverse transcription reaction was performed by using Transcriptor First Strand cDNA Synthesis Kit (Roche, 4897030001), whereby a cDNA was created. By using this cDNA as a template for PCR, a GL4 gene and, as a control, a GAPDH gene were subjected to PCR by the Taqman probe method using ABI 7900HT Fast (ABI, Inc.), and the levels of mRNAs of the respective genes amplified by the PCR were measured. Then, a semi-quantitative level of the mRNA of GL4 was calculated using the level of the amplified mRNA of GAPDH as an internal control. In the measurement of GAPDH, Taqman probe Hs99999905 ml (ABI, Inc.) was used. In the measurement of GL4, the probe #20 in the universal probe library (Roche, 04686934001), and as the amplification primers, a DNA having a base sequence represented by SEQ ID NO: 77 (forward primer) and a DNA having a base sequence represented by SEQ ID NO: 78 (reverse primer) were used. Further, in the negative control group, the level of the mRNA of GL4 and the level of the amplified mRNA of GAPDH were measured in the same manner, respectively, and a semi-quantitative level of the mRNA of GL4 was calculated using the level of the amplified mRNA of GAPDH as an internal control.

The level of the target mRNA in the siRNA-introduced sample was expressed as a relative ratio when the level of the mRNA of GL4 in the group in which the siRNA was not introduced (negative control group) was taken as 1.

An IC₅₀ value was calculated by the logit method. A statistical analysis was performed using statistical analysis software SAS (Release 9.2, SAS Institute Inc.)

In Table 6, the C %, values in the case of 874-BrdA, 874-A, and 874-U are shown.

TABLE 6

| siRNA | 874-U | 874-A | 874-BrdA |
|---|---|---|---|
| IC₅₀ (pmol/L) | 3.3 | 13.3 | 1.8 |

From the results of the above Test Examples 1 and 2, it is found that the siRNA having 8-bromo-2′-deoxyadenosine monophosphate at an end shows a higher knockdown activity against the expression of luciferase than the siRNA having a natural nucleotide.

### Test Example 3

### RNAi Activity of D-Glyceraldehyde-3-Phosphate Dehydrogenase (GAPDH)-Targeting siRNA

The RNAi activity of a GAPDH-targeting siRNA having 8-bromo-2′-deoxyadenosine monophosphate at the 5′ end of the antisense strand was evaluated by measuring an inhibitory effect on the expression of the mRNA of GAPDH as described below.

In a culture dish (Multidish 24 wells, Cat. No. 142475, manufactured by Nunc, Inc.), human cervical cancer-derived cell line Hela cells (CCL-2, purchased from ATCC) were suspended in RPMI medium (Invitrogen, 11875093) containing 10% fetal bovine serum, and the resulting cell suspension was inoculated into each well at 500 μL/well to give 50000 cells/well. Thereto, a solution of an siRNA-Lipofectamine RNAiMAX (Invitrogen, 13778-075) complex mixed in OPTI-MEM (Invitrogen, 31985-070) was added at 100 μL/well, whereby the siRNA was introduced into the Hela cells. The final concentration of the siRNA was set to one value: 100 pmol/L. Further, as a negative control group, cells to which only RNAi MAX was added were inoculated.

The cells after introduction of the siRNA were cultured under the conditions of 37° C. and 5% CO₂ for 24 hours. In order to collect RNA, an RNA extraction kit (RNeasy 74106) of Qiagen, Inc. was used. The cells after culture were washed once with a phosphate buffer, and then lysed with RLT buffer (attached to RNeasy) and collected. Then, the total RNA was collected according to the protocol attached to the kit. By using the total RNA (1 μg) as a template, a reverse transcription reaction was performed using Transcriptor First Strand cDNA Synthesis Kit (Roche, 4897030001), whereby a cDNA was created. By using this cDNA as a template for PCR, a GAPDH gene and, as a control, a PPIB (pepytidyl-prolyl cis-trans isomerase B) gene were subjected to PCR by the Taqman probe method using ABI 7900HT Fast (ABT, Inc.), and the levels of mRNAs of the respective genes amplified by the PCR were measured. Then, a semi-quantitative level of the mRNA of GAPDH was calculated using the level of the amplified mRNA of PPIB as an internal control. In the measurement of GAPDH, Taqman probe Hs999999905 ml (ABI, Inc.) was used. In the measurement of PPIB, Hs01018502 ml (ABI, Inc.) was used. Further, in the negative control group, the level of the mRNA of GAPDH and the level of the amplified mRNA of PPIB were measured in the same manner, respectively, and a semi-quantitative level of the mRNA of GAPDH was calculated using the level of the amplified mRNA of PPIB as an internal control.

The level of the target mRNA in the siRNA-introduced sample was expressed as a relative ratio when the level of the mRNA of GAPDH in the group in which the siRNA was not introduced (negative control group) was taken as 1.

As the siRNA, 217-BrdA, 278-BrdA, 516-BrdA, 624-BrdA, 715-BrdA, 816-BrdA, 936-BrdA, 1096-BrdA, and 1134-BrdA shown in Table 2 were used, and for comparison, a test was performed in the same manner also for siRNAs having adenosine monophosphate at a position corresponding to that of 8-bromo-2′-deoxyadenosine monophosphate of each siRNA (Table 7).

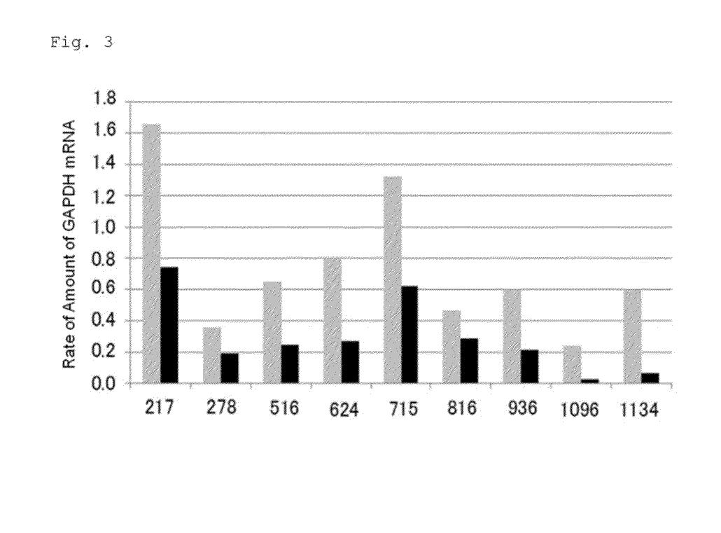

The results of this test are shown in Table 8 and FIG. 3. From these results, it is found that in the case of the siRNA into which 8-bromo-2′-deoxyadenosine monophosphate was introduced at the 5′ end of the antisense strand, the expression level of GAPDH was decreased as compared with the case of the siRNA into which 8-bromo-2′-deoxyadenosine monophosphate was not introduced.

### [Table 7]

TABLE 7

| | sense strand | | antisense strand | |
|---|---|---|---|---|
| Oligo- | sequence | sequence | sequence | sequence |
| nucleotide | (5′→3′) | number | (5′→3′) | number |
| 217-BrdA | GCGCCUGGUCACC | 13 | XGCCCUGGUGA | 14 |
| | AGGGCUGC | | CCAGGCGCCC | |
| 217-A | GCGCCUGGUCACC | 49 | AGCCCUGGUGA | 50 |
| | AGGGCUGC | | CCAGGCGCCC | |
| 278-BrdA | CCCUUCAUUGACC | 15 | XGUUGAGGUCA | 16 |
| | UCAACUAC | | AUGAAGGGGU | |
| 278-A | CCCUUCAUUGACC | 51 | AGUUGAGGUCA | 52 |
| | UCAACUAC | | AUGAAGGGGU | |
| 516-BrdA | GAGCCAAAAGGGU | 17 | XUGAUGACCCU | 18 |
| | CAUCAUCU | | UUUGGCUCCC | |
| 516-A | GAGCCAAAAGGGU | 53 | AUGAUGACCCU | 54 |
| | CAUCAUCU | | UUUGGCUCCC | |
| 624-BrdA | CCUGCACCACCAA | 19 | XAGCAGUUGGU | 20 |
| | CUGCUUAG | | GGUGCAGGAG | |
| 624-A | CCUGCACCACCAA | 55 | AAGCAGUUGGU | 56 |
| | CUGCUUAG | | GGUGCAGGAG | |
| 715-BrdA | CACUGCCACCCAG | 21 | XGUCUUCUGGG | 22 |
| | AAGACUGU | | UGGCAGUGAU | |
| 715-A | CACUGCCACCCAG | 57 | AGUCUUCUGGG | 58 |
| | AAGACUGU | | UGGCAGUGAU | |
| 816-BrdA | AGGCUGUGGGCAA | 23 | XUGACCUUGCC | 24 |
| | GGUCAUCC | | CACAGCCUUG | |
| 816-A | AGGCUGUGGGCAA | 59 | AUGACCUUGCC | 60 |
| | GGUCAUCC | | CACAGCCUUG | |
| 936-BrdA | AUGAUGACAUCAA | 25 | XCCUUCUUGAU | 26 |
| | GAAGGUGG | | GUCAUCAUAU | |
| 936-A | AUGAUGACAUCAA | 61 | ACCUUCUUGAU | 62 |
| | GAAGGUGG | | GUCAUCAUAU | |
| 1096-BrdA | CAAGCUCAUUUCC | 27 | XUACCAGGAAA | 28 |
| | UGGUAUGA | | UGAGCUUGAC | |
| 1096-A | CAAGCUCAUUUCC | 63 | AUACCAGGAAA | 64 |
| | UGGUAUGA | | UGAGCUUGAC | |
| 1134-BrdA | GCAACAGGGUGGU | 29 | XGGUCCACCAC | 30 |
| | GGACCUCA | | CCUGUUGCUG | |
| 1134-A | GCAACAGGGUGGU | 65 | AGGUCCACCAC | 66 |
| | GGACCUCA | | CCUGUUGCUG | |

TABLE 8

| | amount of |
|---|---|
| siRNA | target mRNA |
| 217-BrdA | 0.743 |
| 217-A | 1.654 |
| 278-BrdA | 0.189 |
| 278-A | 0.361 |
| 516-BrdA | 0.246 |
| 516-A | 0.648 |
| 624-BrdA | 0.273 |
| 624-A | 0.798 |
| 715-BrdA | 0.627 |
| 715-A | 1.321 |
| 816-BrdA | 0.291 |
| 816-A | 0.464 |
| 936-BrdA | 0.217 |
| 936-A | 0.602 |
| 1096-BrdA | 0.027 |
| 1096-A | 0.241 |
| 1134-BrdA | 0.067 |
| 1134-A | 0.597 |

### Test Example 4

### Evaluation of Cell Proliferation Inhibitory Activity

The cell proliferation inhibitory activity of a luciferase-targeting siRNA having 8-bromo-2′-deoxyadenosine monophosphate at the 5′ end of the antisense strand was evaluated by measuring an intracellular ATP level and determining cell viability as described below.

In a culture dish (Assay plate, 96-well, with Lid, Cat. No. 3917, manufactured by Costar Co., Ltd.), human cervical cancer-derived cell line Hela cells (CCL-2, purchased from ATCC) transfected with a luciferase expression vector were suspended in RPMI medium (Invitrogen, 11875093) containing 10% fetal bovine serum, and the resulting cell suspension was inoculated into each well at 50 μL/well to give 2000 cells/well (for culturing for 5 days) or 5000 cells/well (for culturing for 2 days). Thereto, a solution of an siRNA-Lipofectamine RNAiMAX (Invitrogen, 13778-075) complex mixed in OPTI-MEM (Invitrogen, 31985-070) was added at 10 L/well, whereby the siRNA was introduced into the Hela cells. The final concentration of the siRNA was set to 100 pmol/L and 10 pmol/L, and N was set to 3. Further, as a negative control group, cells to which only RNAi MAX was added were inoculated.

The cells after introduction of the siRNA were cultured under the conditions of 37° C. and 5% CO₂ for 2 days or 5 days.

CellTiter-Glo™ Luminescent Cell Viability Assay (Promega, G7572), which is a commercially available cell count reagent, and with which an intracellular ATP level only in viable cells among the cells after culture can be measured, was used, and according to the protocol attached to the reagent, the reagent was added to each well at 40 μL/well, followed by incubation for 10 minutes. Then, the amount of luminescence (cps) per second in each well was measured using ARVO (PerkinElmer) according to the protocol.

Simultaneously with the measurement of the amount of luminescence in the luciferase-targeting siRNA treated group, the measurement of the amount of luminescence in the negative control group was performed, and the number cells in each of the siRNA-introduced samples was expressed as a relative ratio when the amount of luminescence in the group in which the siRNA was not introduced (negative control group) was taken as 1.

In this test, as the siRNA, 239-BrdA, 874-BrdA, 904-BrdA, 1084-BrdA, 1203-BrdA, and 1556-BrdA shown in Table 1 were used in the same manner as in Test Example 1, and for comparison, a test was performed in the same manner also for siRNAs having uridine monophosphate or adenosine monophosphate at a position corresponding to that of 8-bromo-2′-deoxyadenosine monophosphate of each siRNA. As the siRNA having adenosine monophosphate, 239-A, 874-A, 904-A, 1084-A, 1203-A, and 1556-A shown in Table 3 were used, and as the siRNA having uridine monophosphate, 239-U, 874-U, 904-U, 1084-U, 1203-U, and 1556-U shown in Table 9 were used.

The results obtained by the above-described methods are shown in Tables 10 to 13.

TABLE 9

| | sense strand | | antisense strand | |
|---|---|---|---|---|
| oligo- | sequence | sequence | sequence | sequence |
| nucleotide | (5′→3′) | number | (5′→3′) | number |
| 239-U | UGCAGCGAGAAUA | 67 | UCAAGCUAUUC | 68 |
| | GCUUGAAG | | UCGCUGCACA | |
| 874-U | UAGCUUCUUCGCU | 47 | UCUCUUAGCGA | 48 |
| | AAGAGAAC | | AGAAGCUAAA | |
| 904-U | CAAGUACGACCUA | 69 | UUUGCUUAGGU | 70 |
| | AGCAAAUU | | CGUACUUGUC | |
| 1084-U | AGGCAAGGUGGUG | 71 | UUAGCCUCGGG | 72 |
| | AGGCUAAAA | | GUUGUUAACG | |
| 1203-U | UUAACAACCCCGA | 73 | UUAGCCUCGGG | 74 |
| | GGCUAAAA | | GUUGUUAACG | |
| 1556-U | GACGAGGUGCCUA | 75 | UUCCUUUAGGC | 76 |
| | AAGGAAUG | | ACCUCGUCCA | |

TABLE 10
Rate of Cell Viability (100 pmol/L, day2)

| antisense strand | | | | | | |
|---|---|---|---|---|---|---|
| 5′ end | 239 | 874 | 904 | 1084 | 1203 | 1556 |
| U | 0.998 | 0.910 | 0.918 | 0.967 | 1.013 | 0.941 |
| A | 0.977 | 0.918 | 0.936 | 0.884 | 0.924 | 0.869 |
| BrdA | 0.948 | 0.989 | 0.962 | 0.930 | 0.931 | 0.886 |

TABLE 11
Rate of Cell Viability (100 pmol/L, day5)

| antisense strand | | | | | | |
|---|---|---|---|---|---|---|
| 5′ end | 239 | 874 | 904 | 1084 | 1203 | 1556 |
| U | 1.024 | 0.998 | 1.011 | 1.039 | 1.012 | 0.992 |
| A | 0.977 | 1.000 | 1.036 | 1.049 | 1.059 | 1.028 |
| BrdA | 1.039 | 1.001 | 1.015 | 1.004 | 0.984 | 0.985 |

TABLE 12
Rate of Cell Viability (10 pmol/L, day2)

| antisense strand | | | | | | |
|---|---|---|---|---|---|---|
| 5′ end | 239 | 874 | 904 | 1084 | 1203 | 1556 |
| U | 0.997 | 0.931 | 0.935 | 0.966 | 1.045 | 0.962 |
| A | 1.004 | 0.923 | 0.973 | 0.935 | 0.983 | 0.932 |
| BrdA | 0.971 | 0.978 | 0.882 | 0.958 | 0.868 | 0.947 |

TABLE 13
Rate of Cell Viability (10 pmol/L, day5)

| antisense strand | | | | | | |
|---|---|---|---|---|---|---|
| 5′ end | 239 | 874 | 904 | 1084 | 1203 | 1556 |
| U | 1.023 | 0.959 | 0.961 | 0.978 | 1.001 | 1.027 |
| A | 1.003 | 1.074 | 1.049 | 1.001 | 1.056 | 1.059 |
| BrdA | 1.019 | 0.994 | 0.975 | 0.972 | 0.959 | 1.016 |

From the above results, it was found that the siRNA, into which 8-bromo-2′-deoxyadenosine monophosphate was introduced at the 5′ end of the antisense strand, and the siRNA having uridine monophosphate or adenosine monophosphate showed almost no cell proliferation inhibition. Further, almost no difference in cell viability was observed between the siRNA, into which 8-bromo-2′-deoxyadenosine monophosphate was introduced, and the siRNA having uridine monophosphate or adenosine monophosphate. From the above results, the siRNA into which 8-bromo-2′-deoxyadenosine monophosphate was introduced did not show cell proliferation inhibitory activity, and thus it was shown that the safety concern with respect to this point is low.

Further, from the results of Test Examples 1 to 3, it was found that the siRNA into which 8-bromo-2′-deoxyadenosine monophosphate was introduced at the 5′ end of the antisense strand had an improved knockdown activity against the target mRNA. From these results, it is considered that the siRNA having 8-bromo-2′-deoxyadenosine monophosphate introduced at the 5′ end of the antisense strand has an improved affinity for AGO2.

As described above, an siRNA can selectively interfere with the expression of any protein through cleavage of an mRNA, and therefore, the application thereof to pharmaceuticals has been expected. From the above-described test results, it is expected to obtain an siRNA having an improved activity by selecting an mRNA involved in the expression of a target protein for treatment, and introducing a modified oligonucleotide at the 5′ end of the siRNA corresponding thereto.

The oligonucleotide of the present invention can be administered alone. However, usually, the oligonucleotide of the present invention is preferably provided in various pharmaceutical preparations. Further, these pharmaceutical preparations are used for animals and humans.

The pharmaceutical preparations according to the present invention can contain, as the active ingredient, the oligonucleotide of the present invention alone or as a mixture with any other active ingredient for treatment. Further, these pharmaceutical preparations are prepared by mixing the active ingredient with one or more pharmaceutically acceptable carriers and then subjecting the mixture to any method well-known in the technical field of pharmaceutics.

As for the administration route, it is preferred to select the most effective route of administration in the treatment. Examples of the administration route include oral administration and parenteral administration such as intravenous administration.

Examples of the dosage form include tablets, injections, and the like.

Suitable dosage forms for the oral administration, for example, tablets, can be prepared by using excipients such as lactose, disintegrators such as starch, lubricants such as magnesium stearate, binders such as hydroxypropyl cellulose, and the like.

Suitable dosage forms for the parenteral administration, for example, injections, can be prepared by using a salt solution, a glucose solution, or a mixed liquid of a salt solution and a glucose solution, and the like.

The doses and the frequencies of administration of the oligonucleotide of the present invention may vary depending upon dosage form, age and body weight of a patient, nature or seriousness of the symptom to be treated, and the like. However, in the parenteral administration such as intravenous administration, in general, a dose of 0.001 to 100 mg, preferably, 0.01 to 10 mg, is administered to an adult patient once or several times a day. However, these doses and frequencies of administration vary according to the various conditions described above.

Hereinafter, embodiments of the present invention will be described with reference to Examples and Reference Examples. Unless otherwise specifically stated, starting materials and reagents used are obtained as commercially available products, or according to known methods. Additionally, the present invention is not limited to these Examples and Reference Examples.

### EXAMPLES

### (1) Synthesis of Oligonucleotide

The synthesis of an oligonucleotide was performed on a scale of 0.5 μmol using a nucleic acid synthesizer: UFPS, Ultra Fast Parallel Synthesizer manufactured by Sigma Co., Ltd. As a solid-phase support, CPG 500 angstrom, rA.rG(tac), SAFC-PROLIGO was used. DMT-2′-O-TBDMS-rA(tac) amidite (SAFC-PROLIGO), DMT-2′-O-TBDMS-rG(tac) amidite (SAFC-PROLIGO), DMT-2′-O-TBDMS-rC (tac) amidite (SAFC-PROLIGO), and DMT-2′-O-TBDMS-rU amidite (SAFC-PROLIGO) were prepared into a 0.1 mol/L acetonitrile solution, 8-Br-dA-CE Phosphoramidite (Glen Research Corporation) was prepared into a 0.1 mol/L acetonitrile solution, Chemical Phosphorylation Reagent II (Glen Research Corporation) was prepared into a 0.06 mol/L acetonitrile solution, and the thus prepared solutions were used for a condensation reaction. As an activator for phosphoramidites, 5-benzylthio-1H-tetrazole (SAFC-PROLIGO) was used, and the condensation time was set to 10 minutes in each case. After synthesis in trityl-off, the resulting product was immersed in a 28% ammonia solution, and then left at 55° C. for 4 hours. After concentration under reduced pressure was performed, 31% triethylamine trihydrofluoride was added thereto, and the resulting mixture was left at 65° C. for 3 hours. Thereafter, 1-butanol was added thereto to stop the reaction. The resulting product was purified by reverse-phase liquid chromatography (SHISEIDO, CAPSELL PAK C18, SG300, 6.0 mm×75 mm, 5% acetonitrile/0.1% triethylammonium acetate buffer, B solution: 50% acetonitrile/water gradient), whereby a target oligonucleotide was obtained.

### (2) Annealing

The single-stranded oligonucleotide obtained in the above (1) was dissolved in a mixed buffer [100 mmol/L potassium acetate, 30 mmol/L 2-[4-(2-hydroxyethyl)-1-piperazinyl]ethanesulfonic acid (HEPES)-KOH (pH 7.4), and 2 mmol/L magnesium acetate] to give a concentration of 50 μmol/L. Equal amounts of sense and antisense strands were mixed with each other and the resulting mixture was left to stand at 80° C. for 10 minutes. The temperature of the mixture was gradually decreased, and the mixture was left to stand at 37° C. for 1 hour, whereby a double-stranded oligonucleotide was obtained.

### INDUSTRIAL APPLICABILITY

According to the present invention, an oligonucleotide having an unnatural nucleotide residue or an unnatural nucleoside residue introduced at the 5′ end for improving the affinity for AGO2, and the like are provided.

### SEQUENCE LISTING FREE TEXT

[Sequence Listing]

1. An oligonucleotide, which has a nucleotide residue or a nucleoside residue represented by formula (I) at the 5′ end thereof, wherein the nucleotide residue or the nucleoside residue binds to an adjacent nucleotide residue via the oxygen atom at position 3: 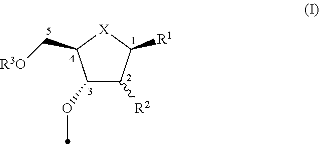
{wherein X represents an oxygen atom, a sulfur atom, a selenium atom, or NR⁴ (wherein R⁴ represents a hydrogen atom, optionally substituted lower alkyl, optionally substituted lower alkenyl, optionally substituted lower alkynyl, optionally substituted lower alkanoyl, optionally substituted lower alkylsulfonyl, optionally substituted aralkyl, optionally substituted aryl, optionally substituted aroyl, or optionally substituted aromatic heterocyclic carbonyl),
R¹ represents formula (II): 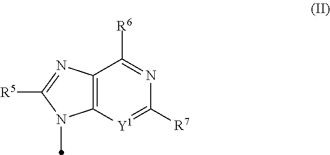
{wherein Y¹ represents a nitrogen atom or CR⁸ [wherein R⁸ represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkanoyl, optionally substituted lower alkylthio, optionally substituted aryl, optionally substituted aralkyl, an optionally substituted aromatic heterocyclic group, optionally substituted aromatic heterocyclic alkyl, —NR^{9a}R^{9b} (wherein R^{9a} and R^{9b} may be the same or different, and each represents a hydrogen atom or optionally substituted lower alkyl), or —CONR^{9c}R^{9d} (wherein R^{9c} and R^{9d} may be the same or different, and each represents a hydrogen atom or optionally substituted lower alkyl)],
R⁵ represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkylthio, optionally substituted lower alkanoyl, optionally substituted aryl, an optionally substituted aromatic heterocyclic group, —NR^{10a}R^{10b} (wherein R^{10a} and R^{10b} have the same definitions as R^{9a} and R^{9b} described above, respectively), or —CONR^{10c}R^{10d} (wherein R^{10c} and R^{10d} have the same definitions as R^{9c} and R^{9d} described above, respectively),
R⁶ and R⁷ may be the same or different, and each represents a hydrogen atom, halogen, cyano, carboxy, optionally substituted lower alkyl, optionally substituted lower alkenyl, optionally substituted lower alkynyl, optionally substituted lower alkoxy, optionally substituted lower alkylthio, optionally substituted lower alkanoyl, optionally substituted lower alkoxycarbonyl, optionally substituted aryl, optionally substituted aralkyl, optionally substituted aryloxy, optionally substituted arylthio, optionally substituted aroyl, an optionally substituted aromatic heterocyclic group, optionally substituted aromatic heterocyclic alkyl, optionally substituted aromatic heterocyclicoxy, optionally substituted aromatic heterocyclicthio, optionally substituted aromatic heterocyclic carbonyl, —NR^{11a}R^{11b} (wherein R^{11a} and R^{11b} may be the same or different, and each represents a hydrogen atom, optionally substituted lower alkyl, optionally substituted lower alkenyl, optionally substituted lower alkynyl, optionally substituted aryl, optionally substituted aralkyl, optionally substituted aromatic heterocyclic alkyl, optionally substituted lower alkanoyl, optionally substituted lower alkylsulfonyl, optionally substituted aroyl, optionally substituted lower arylsulfonyl, an optionally substituted aromatic heterocyclic group, optionally substituted aromatic heterocyclic carbonyl, or optionally substituted aromatic heterocyclic sulfonyl), —CONR^{11c}R^{11d} (wherein R^{11c} and R^{11d} may be the same or different, and each represents a hydrogen atom, optionally substituted lower alkyl, optionally substituted aryl, or an optionally substituted aromatic heterocyclic group), —NHCONR^{11e}R^{11f} (wherein R^{11e} and R^{11f} may be the same or different, and each represents a hydrogen atom, optionally substituted lower alkyl, optionally substituted aralkyl, optionally substituted aromatic heterocyclic alkyl, optionally substituted aryl, or an optionally substituted aromatic heterocyclic group), or —NHCO₂R^{11g} (wherein R^{11g} represents optionally substituted lower alkyl, optionally substituted aralkyl, optionally substituted aromatic heterocyclic alkyl, optionally substituted aryl, or an optionally substituted aromatic heterocyclic group), provided that when Y¹ is a nitrogen atom, R⁵ is a hydrogen atom, R⁶ is —NR^{11a}R^{11b}, and R⁷ is a hydrogen atom, R^{11a} and R^{11b} are not a hydrogen atom at the same time), formula (III): 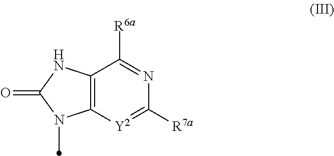
(wherein Y², R^{6a}, and R^{7a} have the same definitions as Y¹, R⁶, and R⁷ described above, respectively), or formula (IV): 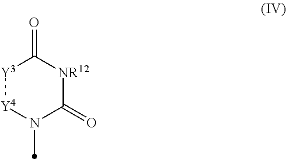
[wherein R¹² represents a hydrogen atom, optionally substituted lower alkyl, optionally substituted lower alkenyl, optionally substituted lower alkynyl, optionally substituted aralkyl, optionally substituted aromatic heterocyclic alkyl, optionally substituted lower alkanoyl, or optionally substituted lower alkylsulfonyl,
--- represents a single bond or a double bond,
provided that when --- represents a single bond,
Y³ represents NR^{13a} (wherein R^{13a} represents a hydrogen atom, optionally substituted lower alkyl, optionally substituted lower alkenyl, optionally substituted lower alkynyl, optionally substituted aralkyl, optionally substituted aromatic heterocyclic alkyl, optionally substituted lower alkanoyl, or optionally substituted lower alkylsulfonyl), or CR^{14a}R^{14b} (wherein R^{14a} and R^{14b} may be the same or different, and each represents a hydrogen atom, halogen, cyano, carboxy, optionally substituted lower alkyl, optionally substituted lower alkenyl, optionally substituted lower alkynyl, optionally substituted lower alkoxy, optionally substituted lower alkoxycarbonyl, optionally substituted lower alkanoyl, optionally substituted lower alkylthio, optionally substituted lower alkylamino, optionally substituted di-lower alkylamino, optionally substituted lower alkylcarbamoyl, optionally substituted di-lower alkylcarbamoyl, optionally substituted aryl, optionally substituted aralkyl, an optionally substituted aromatic heterocyclic group, or optionally substituted aromatic heterocyclic alkyl), and
Y⁴ represents NR^{13b} (wherein R^{13b} has the same definition as R^{13a} described above) or CR^{14c}R^{14d} (wherein R^{14c} and R^{14d} have the same definitions as R^{14a} and R^{14b} described above, respectively),
provided that when --- represents a double bond,
Y³ represents a nitrogen atom or CR^{14e} (wherein R^{14e} has the same definition as R^{14a} described above) and
Y⁴ represents a nitrogen atom or CR^{14f} (wherein R^{14f} has the same definition as R^{14a} described above), but R¹² is a hydrogen atom, and when Y³ is CR^{14e} and Y⁴ is CR^{14f}, R^{14e} and R^{14f} are not a hydrogen atom at the same time],
R² represents a hydrogen atom, hydroxy, halogen, or optionally substituted lower alkoxy, and
R³ represents a hydrogen atom or 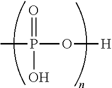
(wherein n represents 1, 2, or 3)).

2. The oligonucleotide according to claim 1, wherein X is an oxygen atom.

3. The oligonucleotide according to claim 1, wherein R¹ is formula (II).

4. The oligonucleotide according to claim 3, wherein Y¹ is a nitrogen atom.

5. The oligonucleotide according to claim 3, wherein R⁵ is halogen, optionally substituted lower alkyl, optionally substituted lower alkenyl, optionally substituted lower alkynyl, optionally substituted lower alkoxy, optionally substituted lower alkanoyl, optionally substituted aryl, an optionally substituted aromatic heterocyclic group, —NR^{10a}R^{10b} (wherein R^{10a} and R^{10b} have the same definitions as described above, respectively), or —CONR^{10c}R^{10d} (wherein R^{10c} and R^{10d} have the same definitions as described above, respectively); and R⁶ is a hydrogen atom.

6. The oligonucleotide according to claim 3, wherein R⁵ is halogen; and R⁶ is a hydrogen atom.

7. The oligonucleotide according to claim 3, wherein R⁵ is a hydrogen atom, halogen, optionally substituted lower alkyl, optionally substituted lower alkenyl, optionally substituted lower alkynyl, optionally substituted lower alkoxy, optionally substituted lower alkanoyl, optionally substituted aryl, an optionally substituted aromatic heterocyclic group, —NR^{10a}R^{10b} (wherein R^{10a} and R^{10b} have the same definitions as described above, respectively), or —CONR^{10c}R^{10d} (wherein R^{10c} and R^{10d} have the same definitions as described above, respectively); and R⁶ is optionally substituted lower alkyl, optionally substituted lower alkenyl, optionally substituted lower alkynyl, optionally substituted aryl, an optionally substituted aromatic heterocyclic group, or —NR^{11a}R^{11b} (wherein R^{11a} and R^{11b} have the same definitions as described above, respectively).

8. The oligonucleotide according to claim 3, wherein R⁵ is a hydrogen atom, halogen, optionally substituted lower alkyl, optionally substituted lower alkenyl, optionally substituted lower alkynyl, optionally substituted lower alkoxy, optionally substituted lower alkanoyl, optionally substituted aryl, an optionally substituted aromatic heterocyclic group, —NR^{10a}R^{10b} (wherein R^{10a} and R^{10b} have the same definitions as described above, respectively), or —CONR^{10c}R^{10d} (wherein R^{10c} and R^{10d} have the same definitions as described above, respectively); and R⁶ is —NR^{11a}R^{11b} (wherein R^{11a} and R^{11b} have the same definitions as described above, respectively).

9. The oligonucleotide according to claim 3, wherein R⁵ is a hydrogen atom, halogen, optionally substituted lower alkyl, optionally substituted lower alkenyl, optionally substituted lower alkynyl, optionally substituted lower alkoxy, optionally substituted lower alkanoyl, optionally substituted aryl, an optionally substituted aromatic heterocyclic group, NR^{10a}R^{10b} (wherein R^{10a} and R^{10b} have the same definitions as described above, respectively), or —CONR^{10c}R^{10d} (wherein R^{10c} and R^{10d} have the same definitions as described above, respectively); and R⁶ is amino.

10. The oligonucleotide according to claim 3, wherein R⁷ is a hydrogen atom.

11. The oligonucleotide according to claim 1, wherein R¹ is formula (III).

12. The oligonucleotide according to claim 1, wherein R¹ is formula (IV).

13. The oligonucleotide according to claim 12, wherein R¹² is a hydrogen atom.

14. The oligonucleotide according to claim 13, wherein --- is a double bond, Y³ is CR^{14e} (wherein R^{14e} has the same definition as described above), and Y⁴ is CR^{14f} (wherein R^{14f} has the same definition as described above).

15. The oligonucleotide according to claim 14, wherein either one of R^{14e} and R^{14f} is a hydrogen atom.

16. The oligonucleotide according to claim 1, wherein R³ is a hydrogen atom.

17. The oligonucleotide according to claim 1, wherein R³ is 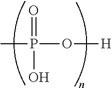
(wherein n has the same definition as described above).

18. The oligonucleotide according to claim 17, wherein n is 1.

19. The oligonucleotide according to claim 17, wherein n is 2.

20. The oligonucleotide according to claim 17, wherein n is 3.

21. An oligonucleotide, which has a nucleotide residue or a nucleoside residue represented by formula (IA) at the 5′ end thereof, wherein the nucleotide residue or the nucleoside residue binds to an adjacent nucleotide residue via the oxygen atom at position 3: 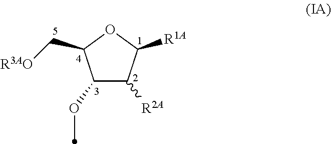
[wherein R^{1A} represents formula (IIA): 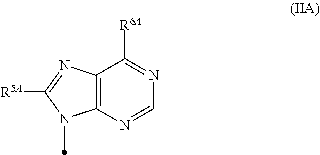
(wherein R^{5A} and R^{6A} have the same definitions as R⁵ and R⁶ described above, respectively, provided that when R⁵ is a hydrogen atom, and R⁶ is —NR^{11a}R^{11b}, R^{11a} and R^{11b} are not a hydrogen atom at the same time), formula (IIIA): 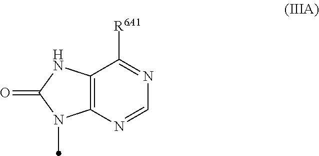
(wherein R^{6A1} has the same definition as R⁶ described above), or formula (IVA): 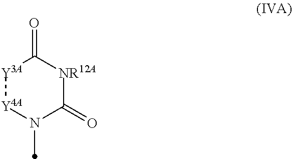
(wherein --- has the same definition as described above, R^{12A}, Y^{3A} and Y^{4A} have the same definitions as R¹², Y³, and Y⁴ described above, respectively),
R^{2A} represents a hydrogen atom, hydroxy, halogen, or optionally substituted lower alkoxy, and
R^{3A} represents a hydrogen atom or 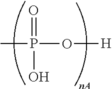
(wherein nA has the same definition as n described above)].

22. The oligonucleotide according to claim 21, wherein R^{1A} is formula (IIA).

23. The oligonucleotide according to claim 22, wherein R^{5A} is halogen, carbamoyl, optionally substituted lower alkyl, optionally substituted lower alkenyl, optionally substituted lower alkynyl, optionally substituted lower alkoxy, optionally substituted lower alkanoyl, optionally substituted lower alkylamino, optionally substituted di-lower alkylamino, optionally substituted lower alkylcarbamoyl, optionally substituted di-lower alkylcarbamoyl, optionally substituted aryl, or an optionally substituted aromatic heterocyclic group; and R^{6A} is a hydrogen atom.

24. The oligonucleotide according to claim 22, wherein R^{5A} is halogen; and R^{6A} is a hydrogen atom.

25. The oligonucleotide according to claim 22, wherein R^{5A} is a hydrogen atom, halogen, carbamoyl, optionally substituted lower alkyl, optionally substituted lower alkenyl, optionally substituted lower alkynyl, optionally substituted lower alkoxy, optionally substituted lower alkanoyl, optionally substituted lower alkylamino, optionally substituted di-lower alkylamino, optionally substituted lower alkylcarbamoyl, optionally substituted di-lower alkylcarbamoyl, optionally substituted aryl, or an optionally substituted aromatic heterocyclic group; and R^{6A} is optionally substituted lower alkyl, optionally substituted lower alkenyl, optionally substituted lower alkynyl, optionally substituted aryl, an optionally substituted aromatic heterocyclic group, or NR^{11a}R^{11b} (wherein R^{11a} and R^{11b} have the same definitions as described above, respectively).

26. The oligonucleotide according to claim 22, wherein R^{6A} is R^{5A} is a hydrogen atom, halogen, carbamoyl, optionally substituted lower alkyl, optionally substituted lower alkenyl, optionally substituted lower alkynyl, optionally substituted lower alkoxy, optionally substituted lower alkanoyl, optionally substituted lower alkylamino, optionally substituted di-lower alkylamino, optionally substituted lower alkylcarbamoyl, optionally substituted di-lower alkylcarbamoyl, optionally substituted aryl, or an optionally substituted aromatic heterocyclic group; and NR^{11a}R^{11b} (wherein R^{11a} and R^{11b} have the same definitions as described above, respectively).

27. The oligonucleotide according to 22, wherein R^{5A} is a hydrogen atom, halogen, carbamoyl, optionally substituted lower alkyl, optionally substituted lower alkenyl, optionally substituted lower alkynyl, optionally substituted lower alkoxy, optionally substituted lower alkanoyl, optionally substituted lower alkylamino, optionally substituted di-lower alkylamino, optionally substituted lower alkylcarbamoyl, optionally substituted di-lower alkylcarbamoyl, optionally substituted aryl, or an optionally substituted aromatic heterocyclic group; and R^{6A} is amino.

28. The oligonucleotide according to claim 21, wherein R^{1A} is formula (IVA).

29. The oligonucleotide according to claim 28, wherein --- is a double bond, Y^{3A} is CR^{14e} (wherein R^{14e} has the same definition as described above), and Y^{4A} is CR^{14f} (wherein R^{14f} has the same definition as described above).

30. The oligonucleotide according to claim 29, wherein R^{14e} and R^{14f} are each a hydrogen atom.

31. The oligonucleotide according to claim 21, wherein R^{3A} is a hydrogen atom.

32. The oligonucleotide according to claim 21, wherein R^{3A} is 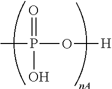
(wherein nA has the same definition as described above).

33. The oligonucleotide according to claim 32, wherein nA is 1.

34. The oligonucleotide according to claim 32, wherein nA is 2.

35. The oligonucleotide according to claim 32, wherein nA is 3.

36. The oligonucleotide according to claim 1, which has a length of 10 to 80 bases.

37. The oligonucleotide according to claim 1, which has a length of 20 to 50 bases.

38. The oligonucleotide according to claim 1, which has a length of 20 to 30 bases.

39. The oligonucleotide according to claim 1, which has a length of 21 to 25 bases.

40. The oligonucleotide according to claim 1, which comprises two oligonucleotide strands.

41. The oligonucleotide according to claim 1, which comprises one oligonucleotide strand.

42. The oligonucleotide according to claim 1, wherein the oligonucleotide is an siRNA.

43. A method for improving the knockdown activity against a target mRNA of an oligonucleotide for treating a disease, comprising:
(i) a step of selecting an oligonucleotide having a knockdown activity against a messenger RNA (mRNA) encoding a protein involved in the disease for treating the disease; and
(ii) a step of introducing a nucleotide residue or a nucleoside residue represented by formula (I) as a nucleotide residue or a nucleoside residue at the 5′ end of the oligonucleotide selected in the above (i): 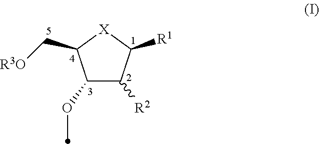
(wherein X, R¹, R², and R³ have the same definitions as described above, respectively).

44. The method for improving the knockdown activity against a target messenger RNA (mRNA) of an oligonucleotide according to claim 43, wherein the oligonucleotide is a small interfering RNA (siRNA).

45. A nucleotide or a salt thereof or a nucleoside or a salt thereof, which is for use in improving the knockdown activity against a target mRNA of an oligonucleotide by being introduced at the 5′ end of the oligonucleotide, and is represented by formula (Ia): 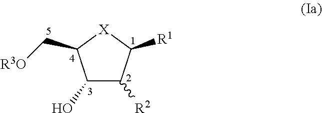
(wherein X, R¹, R², and R³ have the same definitions as described above, respectively).

46. The nucleotide or a salt thereof or a nucleoside or a salt thereof according to claim 45, wherein the oligonucleotide is a small interfering RNA (siRNA).

47. Use of an oligonucleotide for the manufacture of a target protein expression inhibitor, which has a nucleotide residue or a nucleoside residue represented by formula (I) at the 5′ end: 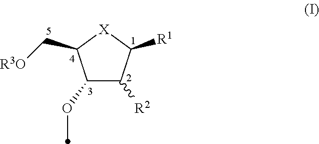
(wherein X, R¹, R², and R³ have the same definitions as described above, respectively).

48. The use according to claim 47, wherein the oligonucleotide is a small interfering RNA (siRNA).


SEQUENCE LISTING (78 sequences)

SEQ ID NO: 1 - RNA, 21 nt, Artificial Sequence
  Synthethic RNA construct
ugcagcgagaauagcuuguag

SEQ ID NO: 2 - RNA, 21 nt, Artificial Sequence
  Synthetic RNA construct
ncaagcuauucucgcugcaca

SEQ ID NO: 3 - RNA, 21 nt, Artificial Sequence
  Synthetic RNA construct
uagcuucuucgcuaagaguac

SEQ ID NO: 4 - RNA, 21 nt, Artificial Sequence
  Synthetic RNA construct
ncucuuagcgaagaagcuaaa

SEQ ID NO: 5 - RNA, 21 nt, Artificial Sequence
  Synthetic RNA construct
caaguacgaccuaagcaauuu

SEQ ID NO: 6 - RNA, 21 nt, Artificial Sequence
  Synthetic RNA construct
nuugcuuaggucguacuuguc

SEQ ID NO: 7 - RNA, 21 nt, Artificial Sequence
  Synthetic RNA construct
aggcaagguggugcccuuuuu

SEQ ID NO: 8 - RNA, 21 nt, Artificial Sequence
  Synthetic RNA construct
naagggcaccaccuugccuac

SEQ ID NO: 9 - RNA, 21 nt, Artificial Sequence
  Synthetic RNA construct
uuaacaaccccgaggcuauaa

SEQ ID NO: 10 - RNA, 21 nt, Artificial Sequence
  Synthetic RNA construct
nuagccucgggguuguuaacg

SEQ ID NO: 11 - RNA, 21 nt, Artificial Sequence
  Synthetic RNA construct
gacgaggugccuaaaggauug

SEQ ID NO: 12 - RNA, 21 nt, Artificial Sequence
  Synthetic RNA construct
nuccuuuaggcaccucgucca

SEQ ID NO: 13 - RNA, 21 nt, Artificial Sequence
  Synthetic RNA construct
gcgccuggucaccagggcugc

SEQ ID NO: 14 - RNA, 21 nt, Artificial Sequence
  Synthetic RNA construct
ngcccuggugaccaggcgccc

SEQ ID NO: 15 - RNA, 21 nt, Artificial Sequence
  Synthetic RNA construct
cccuucauugaccucaacuac

SEQ ID NO: 16 - RNA, 21 nt, Artificial Sequence
  Synthetic RNA construct
nguugaggucaaugaaggggu

SEQ ID NO: 17 - RNA, 21 nt, Artificial Sequence
  Synthetic RNA construct
gagccaaaagggucaucaucu

SEQ ID NO: 18 - RNA, 21 nt, Artificial Sequence
  Synthetic RNA construct
nugaugacccuuuuggcuccc

SEQ ID NO: 19 - RNA, 21 nt, Artificial Sequence
  Synthetic RNA construct
ccugcaccaccaacugcuuag

SEQ ID NO: 20 - RNA, 21 nt, Artificial Sequence
  Synthetic RNA construct
nagcaguugguggugcaggag

SEQ ID NO: 21 - RNA, 21 nt, Artificial Sequence
  Synthetic RNA construct
cacugccacccagaagacugu

SEQ ID NO: 22 - RNA, 21 nt, Artificial Sequence
  Synthetic RNA construct
ngucuucuggguggcagugau

SEQ ID NO: 23 - RNA, 21 nt, Artificial Sequence
  Synthetic RNA construct
aggcugugggcaaggucaucc

SEQ ID NO: 24 - RNA, 21 nt, Artificial Sequence
  Synthetic RNA construct
nugaccuugcccacagccuug

SEQ ID NO: 25 - RNA, 21 nt, Artificial Sequence
  Synthetic RNA construct
augaugacaucaagaaggugg

SEQ ID NO: 26 - RNA, 21 nt, Artificial Sequence
  Synthetic RNA construct
nccuucuugaugucaucauau

SEQ ID NO: 27 - RNA, 21 nt, Artificial Sequence
  Synthetic RNA construct
caagcucauuuccugguauga

SEQ ID NO: 28 - RNA, 21 nt, Artificial Sequence
  Synthetic RNA construct
nuaccaggaaaugagcuugac

SEQ ID NO: 29 - RNA, 21 nt, Artificial Sequence
  Synthetic RNA construct
gcaacagggugguggaccuca

SEQ ID NO: 30 - RNA, 21 nt, Artificial Sequence
  Synthetic RNA construct
ngguccaccacccuguugcug

SEQ ID NO: 31 - RNA, 21 nt, Artificial Sequence
  Synthetic RNA construct
ugcagcgagaauagcuuguag

SEQ ID NO: 32 - RNA, 21 nt, Artificial Sequence
  Synthetic RNA construct
acaagcuauucucgcugcaca

SEQ ID NO: 33 - RNA, 21 nt, Artificial Sequence
  Synthetic RNA construct
uagcuucuucgcuaagaguac

SEQ ID NO: 34 - RNA, 21 nt, Artificial Sequence
  Synthetic RNA construct
acucuuagcgaagaagcuaaa

SEQ ID NO: 35 - RNA, 21 nt, Artificial Sequence
  Synthetic RNA construct
caaguacgaccuaagcaauuu

SEQ ID NO: 36 - RNA, 21 nt, Artificial Sequence
  Synthetic RNA construct
auugcuuaggucguacuuguc

SEQ ID NO: 37 - RNA, 21 nt, Artificial Sequence
  Synthetic RNA construct
aggcaagguggugcccuuuuu

SEQ ID NO: 38 - RNA, 21 nt, Artificial Sequence
  Synthetic RNA construct
aaagggcaccaccuugccuac

SEQ ID NO: 39 - RNA, 21 nt, Artificial Sequence
  Synthetic RNA construct
uuaacaaccccgaggcuauaa

SEQ ID NO: 40 - RNA, 21 nt, Artificial Sequence
  Synthetic RNA construct
auagccucgggguuguuaacg

SEQ ID NO: 41 - RNA, 21 nt, Artificial Sequence
  Synthetic RNA construct
gacgaggugccuaaaggauug

SEQ ID NO: 42 - RNA, 21 nt, Artificial Sequence
  Synthetic RNA construct
auccuuuaggcaccucgucca

SEQ ID NO: 43 - RNA, 21 nt, Artificial Sequence
  Synthetic RNA construct
uagcuucuucgcuaagagcac

SEQ ID NO: 44 - RNA, 21 nt, Artificial Sequence
  Synthetic RNA construct
gcucuuagcgaagaagcuaaa

SEQ ID NO: 45 - RNA, 21 nt, Artificial Sequence
  Synthetic RNA construct
uagcuucuucgcuaagaggac

SEQ ID NO: 46 - RNA, 21 nt, Artificial Sequence
  Synthetic RNA construct
ccucuuagcgaagaagcuaaa

SEQ ID NO: 47 - RNA, 21 nt, Artificial Sequence
  Synthetic RNA construct
uagcuucuucgcuaagagaac

SEQ ID NO: 48 - RNA, 21 nt, Artificial Sequence
  Synthetic RNA construct
ucucuuagcgaagaagcuaaa

SEQ ID NO: 49 - RNA, 21 nt, Artificial Sequence
  Synthetic RNA construct
gcgccuggucaccagggcugc

SEQ ID NO: 50 - RNA, 21 nt, Artificial Sequence
  Synthetic RNA construct
agcccuggugaccaggcgccc

SEQ ID NO: 51 - RNA, 21 nt, Artificial Sequence
  Synthetic RNA construct
cccuucauugaccucaacuac

SEQ ID NO: 52 - RNA, 21 nt, Artificial Sequence
  Synthetic RNA construct
aguugaggucaaugaaggggu

SEQ ID NO: 53 - RNA, 21 nt, Artificial Sequence
  Synthetic RNA construct
gagccaaaagggucaucaucu

SEQ ID NO: 54 - RNA, 21 nt, Artificial Sequence
  Synthetic RNA construct
augaugacccuuuuggcuccc

SEQ ID NO: 55 - RNA, 21 nt, Artificial Sequence
  Synthetic RNA construct
ccugcaccaccaacugcuuag

SEQ ID NO: 56 - RNA, 21 nt, Artificial Sequence
  Synthetic RNA construct
aagcaguugguggugcaggag

SEQ ID NO: 57 - RNA, 21 nt, Artificial Sequence
  Synthetic RNA construct
cacugccacccagaagacugu

SEQ ID NO: 58 - RNA, 21 nt, Artificial Sequence
  Synthetic RNA construct
agucuucuggguggcagugau

SEQ ID NO: 59 - RNA, 21 nt, Artificial Sequence
  Synthetic RNA construct
aggcugugggcaaggucaucc

SEQ ID NO: 60 - RNA, 21 nt, Artificial Sequence
  Synthetic RNA construct
augaccuugcccacagccuug

SEQ ID NO: 61 - RNA, 21 nt, Artificial Sequence
  Synthetic RNA construct
augaugacaucaagaaggugg

SEQ ID NO: 62 - RNA, 21 nt, Artificial Sequence
  Synthetic RNA construct
accuucuugaugucaucauau

SEQ ID NO: 63 - RNA, 21 nt, Artificial Sequence
  Synthetic RNA construct
caagcucauuuccugguauga

SEQ ID NO: 64 - RNA, 21 nt, Artificial Sequence
  Synthetic RNA construct
auaccaggaaaugagcuugac

SEQ ID NO: 65 - RNA, 21 nt, Artificial Sequence
  Synthetic RNA construct
gcaacagggugguggaccuca

SEQ ID NO: 66 - RNA, 21 nt, Artificial Sequence
  Synthetic RNA construct
agguccaccacccuguugcug

SEQ ID NO: 67 - RNA, 21 nt, Artificial Sequence
  Synthetic RNA construct
ugcagcgagaauagcuugaag

SEQ ID NO: 68 - RNA, 21 nt, Artificial Sequence
  Synthetic RNA construct
ucaagcuauucucgcugcaca

SEQ ID NO: 69 - RNA, 21 nt, Artificial Sequence
  Synthetic RNA construct
caaguacgaccuaagcaaauu

SEQ ID NO: 70 - RNA, 21 nt, Artificial Sequence
  Synthetic RNA construct
uuugcuuaggucguacuuguc

SEQ ID NO: 71 - RNA, 21 nt, Artificial Sequence
  Synthetic RNA construct
aggcaagguggugcccuuauu

SEQ ID NO: 72 - RNA, 21 nt, Artificial Sequence
  Synthetic RNA construct
uaagggcaccaccuugccuac

SEQ ID NO: 73 - RNA, 21 nt, Artificial Sequence
  Synthetic RNA construct
uuaacaaccccgaggcuaaaa

SEQ ID NO: 74 - RNA, 21 nt, Artificial Sequence
  Synthetic RNA construct
uuagccucgggguuguuaacg

SEQ ID NO: 75 - RNA, 21 nt, Artificial Sequence
  Synthetic RNA construct
gacgaggugccuaaaggaaug

SEQ ID NO: 76 - RNA, 21 nt, Artificial Sequence
  Synthetic RNA construct
uuccuuuaggcaccucgucca

SEQ ID NO: 77 - DNA, 20 bp, Artificial Sequence
  Synthetic DNA construct
catgaccgagaaggagatcg

SEQ ID NO: 78 - DNA, 19 bp, Artificial Sequence
  Synthetic DNA construct
cagcttcttggcggttgta